SEQUENCE LISTING (316 sequences)

SEQ ID NO: 1 - DNA, 24 bp, Human papillomavirus type 16
cgtagacattcgtactttggaaga

SEQ ID NO: 2 - DNA, 24 bp, Human papillomavirus type 16
attcgtactttggaagacctgtta

SEQ ID NO: 3 - DNA, 24 bp, Human papillomavirus type 16
ttactacttcaactgataccacac

SEQ ID NO: 4 - DNA, 24 bp, Human papillomavirus type 16
ctacttcaactgataccacacctg

SEQ ID NO: 5 - DNA, 24 bp, Human papillomavirus type 16
gtaagtggtgtttggcatatagtg

SEQ ID NO: 6 - DNA, 24 bp, Human papillomavirus type 16
atatttgtaagtggtgtttggcat

SEQ ID NO: 7 - DNA, 30 bp, Human papillomavirus type 16
ggttgtagaagtatctgtaataaagtcatc

SEQ ID NO: 8 - DNA, 30 bp, Human papillomavirus type 16
ttcaggacccacaggagcgacccagaaagt

SEQ ID NO: 9 - DNA, 30 bp, Human papillomavirus type 16
aggacccacaggagcgacccagaaagttac

SEQ ID NO: 10 - DNA, 30 bp, Human papillomavirus type 16
gacccacaggagcgacccagaaagttacca

SEQ ID NO: 11 - DNA, 30 bp, Human papillomavirus type 16
cccacaggagcgacccagaaagttaccaca

SEQ ID NO: 12 - DNA, 41 bp, Human papillomavirus type 16
tgacgagaacgaaaatgacagtgatacaggtgaagatttgg

SEQ ID NO: 13 - DNA, 41 bp, Human papillomavirus type 16; identical to SEQ ID NO: 12

SEQ ID NO: 14 - DNA, 30 bp, Human papillomavirus type 16
acgagaacgaaaatgacagtgatacaggtg

SEQ ID NO: 15 - DNA, 30 bp, Human papillomavirus type 16
cgagaacgaaaatgacagtgatacaggtga

SEQ ID NO: 16 - DNA, 30 bp, Human papillomavirus type 16
gagaacgaaaatgacagtgatacaggtgaa

SEQ ID NO: 17 - DNA, 30 bp, Human papillomavirus type 16
agaacgaaaatgacagtgatacaggtgaag

SEQ ID NO: 18 - DNA, 30 bp, Human papillomavirus type 16
gaacgaaaatgacagtgatacaggtgaaga

SEQ ID NO: 19 - DNA, 30 bp, Human papillomavirus type 16
aacgaaaatgacagtgatacaggtgaagat

SEQ ID NO: 20 - DNA, 41 bp, Human papillomavirus type 16
actggagggcattttacactttcatcatccactattagtac

SEQ ID NO: 21 - DNA, 41 bp, Human papillomavirus type 16; identical to SEQ ID NO: 20

SEQ ID NO: 22 - DNA, 41 bp, Human papillomavirus type 16
aggccagcattaacctctaggcgtactggcattaggtacag

SEQ ID NO: 23 - DNA, 30 bp, Human papillomavirus type 16
aggagattatttgaaagcgaagacagcggg

SEQ ID NO: 24 - DNA, 30 bp, Human papillomavirus type 16
ggagattatttgaaagcgaagacagcgggt

SEQ ID NO: 25 - DNA, 30 bp, Human papillomavirus type 16
gagattatttgaaagcgaagacagcgggta

SEQ ID NO: 26 - DNA, 30 bp, Human papillomavirus type 16
tgcttttgtaaccactcccactaaacttat

SEQ ID NO: 27 - DNA, 30 bp, Human papillomavirus type 16; identical to SEQ ID NO: 26

SEQ ID NO: 28 - DNA, 30 bp, Human papillomavirus type 16
ataacaccttctacatatactaccacttca

SEQ ID NO: 29 - DNA, 30 bp, Human papillomavirus type 16
aaacatacacctccagcacctaaagaagat

SEQ ID NO: 30 - DNA, 30 bp, Human papillomavirus type 16
acatacacctccagcacctaaagaagatga

SEQ ID NO: 31 - DNA, 30 bp, Human papillomavirus type 16
acacctccagcacctaaagaagatgatccc

SEQ ID NO: 32 - DNA, 24 bp, Human papillomavirus type 18
tatcacaccttcgtctacctctgt

SEQ ID NO: 33 - DNA, 24 bp, Human papillomavirus type 18
tgatccgtccattattgaagttcc

SEQ ID NO: 34 - DNA, 24 bp, Human papillomavirus type 18
cattgtcctccgtggcagatacta

SEQ ID NO: 35 - DNA, 24 bp, Human papillomavirus type 18
ttgtcctccgtggcagatactaaa

SEQ ID NO: 36 - DNA, 24 bp, Human papillomavirus type 18
cattgtcctccgtggcagatacta

SEQ ID NO: 37 - DNA, 24 bp, Human papillomavirus type 18
ttgtcctccgtggcagatactaaa

SEQ ID NO: 38 - DNA, 30 bp, Human papillomavirus type 18
cagtggctaaccctgagtttcttacacgtc

SEQ ID NO: 39 - DNA, 30 bp, Human papillomavirus type 18
agtggctaaccctgagtttcttacacgtcc

SEQ ID NO: 40 - DNA, 41 bp, Human papillomavirus type 18
gtggctaaccctgagtttcttacacgtccatcctctttaat

SEQ ID NO: 41 - DNA, 41 bp, Human papillomavirus type 18; identical to SEQ ID NO: 40

SEQ ID NO: 42 - DNA, 30 bp, Human papillomavirus type 18
taaccctgagtttcttacacgtccatcctc

SEQ ID NO: 43 - DNA, 30 bp, Human papillomavirus type 18
aaccctgagtttcttacacgtccatcctct

SEQ ID NO: 44 - DNA, 30 bp, Human papillomavirus type 18
accctgagtttcttacacgtccatcctctt

SEQ ID NO: 45 - DNA, 30 bp, Human papillomavirus type 18
ccctgagtttcttacacgtccatcctcttt

SEQ ID NO: 46 - DNA, 30 bp, Human papillomavirus type 18
cctgagtttcttacacgtccatcctcttta

SEQ ID NO: 47 - DNA, 30 bp, Human papillomavirus type 18
ctgagtttcttacacgtccatcctctttaa

SEQ ID NO: 48 - DNA, 30 bp, Human papillomavirus type 18
tgagtttcttacacgtccatcctctttaat

SEQ ID NO: 49 - DNA, 30 bp, Human papillomavirus type 18
gagtttcttacacgtccatcctctttaatt

SEQ ID NO: 50 - DNA, 30 bp, Human papillomavirus type 18
acatttgatcctcgtagtgatgttcctgat

SEQ ID NO: 51 - DNA, 30 bp, Human papillomavirus type 18; identical to SEQ ID NO: 39

SEQ ID NO: 52 - DNA, 30 bp, Human papillomavirus type 18; identical to SEQ ID NO: 43

SEQ ID NO: 53 - DNA, 30 bp, Human papillomavirus type 18; identical to SEQ ID NO: 45

SEQ ID NO: 54 - DNA, 30 bp, Human papillomavirus type 18; identical to SEQ ID NO: 48

SEQ ID NO: 55 - DNA, 30 bp, Human papillomavirus type 18; identical to SEQ ID NO: 50

SEQ ID NO: 56 - DNA, 24 bp, Human papillomavirus type 31
cagacgttatacctaaaatagaac

SEQ ID NO: 57 - DNA, 24 bp, Human papillomavirus type 31
agacgttatacctaaaatagaaca

SEQ ID NO: 58 - DNA, 24 bp, Human papillomavirus type 31
ctataatttaggtgtcacgccata

SEQ ID NO: 59 - DNA, 24 bp, Human papillomavirus type 31
tataatttaggtgtcacgccatag

SEQ ID NO: 60 - DNA, 24 bp, Human papillomavirus type 31
aacacttgttacatctaaaattgc

SEQ ID NO: 61 - DNA, 24 bp, Human papillomavirus type 31
cacatagttgaactacagttgtat

SEQ ID NO: 62 - DNA, 24 bp, Human papillomavirus type 31
acacatagttgaactacagttgta

SEQ ID NO: 63 - DNA, 41 bp, Human papillomavirus type 31
atatgtccctcttagtacacgtccttctacagtatctgagg

SEQ ID NO: 64 - DNA, 41 bp, Human papillomavirus type 31; identical to SEQ ID NO: 63

SEQ ID NO: 65 - DNA, 30 bp, Human papillomavirus type 31
tatgtccctcttagtacacgtccttctaca

SEQ ID NO: 66 - DNA, 30 bp, Human papillomavirus type 31
atgtccctcttagtacacgtccttctacag

SEQ ID NO: 67 - DNA, 30 bp, Human papillomavirus type 31
tgtccctcttagtacacgtccttctacagt

SEQ ID NO: 68 - DNA, 30 bp, Human papillomavirus type 31
tccctcttagtacacgtccttctacagtat

SEQ ID NO: 69 - DNA, 41 bp, Human papillomavirus type 31
tgtttaaacatgctagtacaactatgctgatgcagtagttc

SEQ ID NO: 70 - DNA, 30 bp, Human papillomavirus type 31
ttaaacatgctagtacaactatgctgatgc

SEQ ID NO: 71 - DNA, 30 bp, Human papillomavirus type 31
tacctattagaccaccagttagcattgacc

SEQ ID NO: 72 - DNA, 30 bp, Human papillomavirus type 31
cctattagaccaccagttagcattgaccct

SEQ ID NO: 73 - DNA, 30 bp, Human papillomavirus type 31
ctattagaccaccagttagcattgaccctg

SEQ ID NO: 74 - DNA, 30 bp, Human papillomavirus type 31
tattagaccaccagttagcattgaccctgt

SEQ ID NO: 75 - DNA, 40 bp, Human papillomavirus type 31
gtttcctgcctaacacaccttgccaacatataatccagtc

SEQ ID NO: 76 - DNA, 40 bp, Human papillomavirus type 31
tttcctgcctaacacaccttgccaacatataatccagtcc

SEQ ID NO: 77 - DNA, 40 bp, Human papillomavirus type 31
ttcctgcctaacacaccttgccaacatataatccagtcca

SEQ ID NO: 78 - DNA, 30 bp, Human papillomavirus type 31
tttcctgcctaacacaccttgccaacatat

SEQ ID NO: 79 - DNA, 24 bp, Human papillomavirus type 33
tattacatctcgtagacatactgt

SEQ ID NO: 80 - DNA, 24 bp, Human papillomavirus type 33
gctattacatctcgtagacatact

SEQ ID NO: 81 - DNA, 24 bp, Human papillomavirus type 33
ctattacatctcgtagacatactg

SEQ ID NO: 82 - DNA, 24 bp, Human papillomavirus type 33
gtaccaataattttttagcgttag

SEQ ID NO: 83 - DNA, 41 bp, Human papillomavirus type 33
tattgtgcctttagaccacaccgtgccaaatgaacaatatg

SEQ ID NO: 84 - DNA, 33 bp, Human papillomavirus type 33
tgcctttagaccacaccgtgccaaatgaacaat

SEQ ID NO: 85 - DNA, 30 bp, Human papillomavirus type 33
gtgcctttagaccacaccgtgccaaatgaa

SEQ ID NO: 86 - DNA, 30 bp, Human papillomavirus type 33
tgcctttagaccacaccgtgccaaatgaac

SEQ ID NO: 87 - DNA, 30 bp, Human papillomavirus type 33
gcctttagaccacaccgtgccaaatgaaca

SEQ ID NO: 88 - DNA, 30 bp, Human papillomavirus type 33
cctttagaccacaccgtgccaaatgaacaa

SEQ ID NO: 89 - DNA, 30 bp, Human papillomavirus type 33
ctttagaccacaccgtgccaaatgaacaat

SEQ ID NO: 90 - DNA, 41 bp, Human papillomavirus type 33
gatataccttcccctttatttcccacatctagcccatttgt

SEQ ID NO: 91 - DNA, 30 bp, Human papillomavirus type 33
taccttcccctttatttcccacatctagcc

SEQ ID NO: 92 - DNA, 30 bp, Human papillomavirus type 33
accttcccctttatttcccacatctagccc

SEQ ID NO: 93 - DNA, 30 bp, Human papillomavirus type 33
tttatttcccacatctagcccatttgttcc

SEQ ID NO: 94 - DNA, 30 bp, Human papillomavirus type 33
tatttcccacatctagcccatttgttccta

SEQ ID NO: 95 - DNA, 30 bp, Human papillomavirus type 33
acagcctttacatgatacttctacatcgtc

SEQ ID NO: 96 - DNA, 30 bp, Human papillomavirus type 33
cagcctttacatgatacttctacatcgtct

SEQ ID NO: 97 - DNA, 30 bp, Human papillomavirus type 33
agcctttacatgatacttctacatcgtctt

SEQ ID NO: 98 - DNA, 30 bp, Human papillomavirus type 33
tttccttttgacaccattgttgtagacggt

SEQ ID NO: 99 - DNA, 24 bp, Human papillomavirus type 35
tgacatccataagtacacatgata

SEQ ID NO: 100 - DNA, 24 bp, Human papillomavirus type 35
gacatccataagtacacatgataa

SEQ ID NO: 101 - DNA, 24 bp, Human papillomavirus type 35
catgttgtaagggttgtaattcta

SEQ ID NO: 102 - DNA, 24 bp, Human papillomavirus type 35
atttaatgatgttgaaacagtggt

SEQ ID NO: 103 - DNA, 51 bp, Human papillomavirus type 35
tgttgaccctgcctttatgacttctcctgcaaaacttattacatatgataa

SEQ ID NO: 104 - DNA, 43 bp, Human papillomavirus type 35
gaccctgcctttatgacttctcctgcaaaacttattacatatg

SEQ ID NO: 105 - DNA, 40 bp, Human papillomavirus type 35
gaccctgcctttatgacttctcctgcaaaacttattacat

SEQ ID NO: 106 - DNA, 40 bp, Human papillomavirus type 35
accctgcctttatgacttctcctgcaaaacttattacata

SEQ ID NO: 107 - DNA, 30 bp, Human papillomavirus type 35
ccctgcctttatgacttctcctgcaaaact

SEQ ID NO: 108 - DNA, 30 bp, Human papillomavirus type 35
cctgcctttatgacttctcctgcaaaactt

SEQ ID NO: 109 - DNA, 30 bp, Human papillomavirus type 35
ctgcctttatgacttctcctgcaaaactta

SEQ ID NO: 110 - DNA, 30 bp, Human papillomavirus type 35
tgcctttatgacttctcctgcaaaacttat

SEQ ID NO: 111 - DNA, 30 bp, Human papillomavirus type 35
gcctttatgacttctcctgcaaaacttatt

SEQ ID NO: 112 - DNA, 40 bp, Human papillomavirus type 35
ccttaaccctgatacaaccttacaatttgagcatgaggat

SEQ ID NO: 113 - DNA, 40 bp, Human papillomavirus type 35
ttaaccctgatacaaccttacaatttgagcatgaggatat

SEQ ID NO: 114 - DNA, 40 bp, Human papillomavirus type 35
aaccctgatacaaccttacaatttgagcatgaggatatta

SEQ ID NO: 115 - DNA, 40 bp, Human papillomavirus type 35
cctgatacaaccttacaatttgagcatgaggatattagct

SEQ ID NO: 116 - DNA, 30 bp, Human papillomavirus type 39
cagtaaggtttagtaggcttggcaaaaagg

SEQ ID NO: 117 - DNA, 24 bp, Human papillomavirus type 39
tagttcacgctgagccctctgatg

SEQ ID NO: 118 - DNA, 24 bp, Human papillomavirus type 39
atgctgtcactagaccgccacata

SEQ ID NO: 119 - DNA, 51 bp, Human papillomavirus type 39
tagatactgcatttaataatacaagggattcgggcactacatataacacag

SEQ ID NO: 120 - DNA, 44 bp, Human papillomavirus type 39
tgcatttaataatacaagggattcgggcactacatataacacag

SEQ ID NO: 121 - DNA, 30 bp, Human papillomavirus type 39
actgcatttaataatacaagggattcgggc

SEQ ID NO: 122 - DNA, 40 bp, Human papillomavirus type 39
actgcatttaataatacaagggattcgggcactacatata

SEQ ID NO: 123 - DNA, 30 bp, Human papillomavirus type 39
tttaataatacaagggattcgggcactaca

SEQ ID NO: 124 - DNA, 30 bp, Human papillomavirus type 39
ttaataatacaagggattcgggcactacat

SEQ ID NO: 125 - DNA, 30 bp, Human papillomavirus type 39
aatacaagggattcgggcactacatataac

SEQ ID NO: 126 - DNA, 30 bp, Human papillomavirus type 39
gtactactccacagttgccattggtgcctt

SEQ ID NO: 127 - DNA, 30 bp, Human papillomavirus type 39
tactactccacagttgccattggtgccttc

SEQ ID NO: 128 - DNA, 30 bp, Human papillomavirus type 39
actactccacagttgccattggtgccttct

SEQ ID NO: 129 - DNA, 30 bp, Human papillomavirus type 39
ctactccacagttgccattggtgccttctg

SEQ ID NO: 130 - DNA, 30 bp, Human papillomavirus type 39
tactccacagttgccattggtgccttctgg

SEQ ID NO: 131 - DNA, 30 bp, Human papillomavirus type 39; identical to SEQ ID NO: 126

SEQ ID NO: 132 - DNA, 30 bp, Human papillomavirus type 39; identical to SEQ ID NO: 127

SEQ ID NO: 133 - DNA, 30 bp, Human papillomavirus type 39; identical to SEQ ID NO: 128

SEQ ID NO: 134 - DNA, 30 bp, Human papillomavirus type 39; identical to SEQ ID NO: 129

SEQ ID NO: 135 - DNA, 30 bp, Human papillomavirus type 39; identical to SEQ ID NO: 130

SEQ ID NO: 136 - DNA, 24 bp, Human papillomavirus type 45
tagggctaatcaacaggtccgtgt

SEQ ID NO: 137 - DNA, 24 bp, Human papillomavirus type 45
taatcaacaggtccgtgtgtccac

SEQ ID NO: 138 - DNA, 24 bp, Human papillomavirus type 45
ctgttattacgcaggatgttaggg

SEQ ID NO: 139 - DNA, 24 bp, Human papillomavirus type 45
ttacgcaggatgttagggataatg

SEQ ID NO: 140 - DNA, 24 bp, Human papillomavirus type 45
tacgcaggatgttagggataatgt

SEQ ID NO: 141 - DNA, 30 bp, Human papillomavirus type 45
aatggtactgtaacattactgtaagaggat

SEQ ID NO: 142 - DNA, 24 bp, Human papillomavirus type 45
actgcttaaacttagtagggtcat

SEQ ID NO: 143 - DNA, 30 bp, Human papillomavirus type 45
tactatactgcttaaacttagtagggtcat

SEQ ID NO: 144 - DNA, 41 bp, Human papillomavirus type 45
agtttttaacacatccctcatcgttggttacatttgataat

SEQ ID NO: 145 - DNA, 30 bp, Human papillomavirus type 45
ttaacacatccctcatcgttggttacattt

SEQ ID NO: 146 - DNA, 30 bp, Human papillomavirus type 45
aacacatccctcatcgttggttacatttga

SEQ ID NO: 147 - DNA, 30 bp, Human papillomavirus type 45
acacatccctcatcgttggttacatttgat

SEQ ID NO: 148 - DNA, 30 bp, Human papillomavirus type 45
cacatccctcatcgttggttacatttgata

SEQ ID NO: 149 - DNA, 41 bp, Human papillomavirus type 45
tgcagacttcccacctcctgcgtccactacacctagcacta

SEQ ID NO: 150 - DNA, 36 bp, Human papillomavirus type 45
tgcagacttcccacctcctgcgtccactacacctag

SEQ ID NO: 151 - DNA, 30 bp, Human papillomavirus type 45
acttcccacctcctgcgtccactacaccta

SEQ ID NO: 152 - DNA, 41 bp, Human papillomavirus type 45
ttgcaggatacaaagtgcgaggttccattagacatttgtca

SEQ ID NO: 153 - DNA, 36 bp, Human papillomavirus type 45
ttgcaggatacaaagtgcgaggttccattagacatt

SEQ ID NO: 154 - DNA, 30 bp, Human papillomavirus type 45
ggatacaaagtgcgaggttccattagacat

SEQ ID NO: 155 - DNA, 30 bp, Human papillomavirus type 45
caccacactatcctttgagcctaccagtaa

SEQ ID NO: 156 - DNA, 30 bp, Human papillomavirus type 45
accacactatcctttgagcctaccagtaat

SEQ ID NO: 157 - DNA, 30 bp, Human papillomavirus type 45
ccacactatcctttgagcctaccagtaatg

SEQ ID NO: 158 - DNA, 30 bp, Human papillomavirus type 45
atccaaagtattccttgaccatgccttcta

SEQ ID NO: 159 - DNA, 30 bp, Human papillomavirus type 45
aaagtattccttgaccatgccttctactgc

SEQ ID NO: 160 - DNA, 30 bp, Human papillomavirus type 45
agtattccttgaccatgccttctactgctg

SEQ ID NO: 161 - DNA, 30 bp, Human papillomavirus type 45
tgttatgggtgacacagtacctacggacct

SEQ ID NO: 162 - DNA, 24 bp, Human papillomavirus type 51
gagagtatagacgttatagcaggt

SEQ ID NO: 163 - DNA, 24 bp, Human papillomavirus type 51
cagtacgctttagtaggttaggtc

SEQ ID NO: 164 - DNA, 24 bp, Human papillomavirus type 51
acggagcttcaattctgtaacacg

SEQ ID NO: 165 - DNA, 24 bp, Human papillomavirus type 51
aacacggagcttcaattctgtaac

SEQ ID NO: 166 - DNA, 24 bp, Human papillomavirus type 51
agtctggaactgcctgcatagtaa

SEQ ID NO: 167 - DNA, 24 bp, Human papillomavirus type 51
attagtctggaactgcctgcatag

SEQ ID NO: 168 - DNA, 41 bp, Human papillomavirus type 51
aaagatgtagtattgcatttaacaccacagactgaaattga

SEQ ID NO: 169 - DNA, 41 bp, Human papillomavirus type 51; identical to SEQ ID NO: 168

SEQ ID NO: 170 - DNA, 30 bp, Human papillomavirus type 51
tgtagtattgcatttaacaccacagactga

SEQ ID NO: 171 - DNA, 30 bp, Human papillomavirus type 51
gtagtattgcatttaacaccacagactgaa

SEQ ID NO: 172 - DNA, 41 bp, Human papillomavirus type 51
cacaccacacctatgtcacactcctctttgtctaggcagtt

SEQ ID NO: 173 - DNA, 41 bp, Human papillomavirus type 51; identical to SEQ ID NO: 172

SEQ ID NO: 174 - DNA, 30 bp, Human papillomavirus type 51
accacacctatgtcacactcctctttgtct

SEQ ID NO: 175 - DNA, 30 bp, Human papillomavirus type 51
cacacctatgtcacactcctctttgtctag

SEQ ID NO: 176 - DNA, 30 bp, Human papillomavirus type 51
tatgcgtgaccagctaccagaaagacgggc

SEQ ID NO: 177 - DNA, 30 bp, Human papillomavirus type 51
tccccacacttccattgacaccaagcattc

SEQ ID NO: 178 - DNA, 30 bp, Human papillomavirus type 51
ccacacttccattgacaccaagcattctat

SEQ ID NO: 179 - DNA, 40 bp, Human papillomavirus type 51
acacttccattgacaccaagcattctattgttatactagg

SEQ ID NO: 180 - DNA, 30 bp, Human papillomavirus type 51
cacttccattgacaccaagcattctattgt

SEQ ID NO: 181 - DNA, 30 bp, Human papillomavirus type 51
ccattgacaccaagcattctattgttatac

SEQ ID NO: 182 - DNA, 24 bp, Human papillomavirus type 56
atccgttatttattgatccccctg

SEQ ID NO: 183 - DNA, 24 bp, Human papillomavirus type 56
cgttatttattgatccccctgtta

SEQ ID NO: 184 - DNA, 24 bp, Human papillomavirus type 56
tacgtgtttgtatagtagcctttc

SEQ ID NO: 185 - DNA, 24 bp, Human papillomavirus type 56
cctctacgtgtttgtatagtagcc

SEQ ID NO: 186 - DNA, 55 bp, Human papillomavirus type 56
taaggtaactgaccctgcatttcttgatagacctgcaacattagtatctgctgat

SEQ ID NO: 187 - DNA, 36 bp, Human papillomavirus type 56
ggtaactgaccctgcatttcttgatagacctgcaac

SEQ ID NO: 188 - DNA, 30 bp, Human papillomavirus type 56
ggtaactgaccctgcatttcttgatagacc

SEQ ID NO: 189 - DNA, 30 bp, Human papillomavirus type 56
gtaactgaccctgcatttcttgatagacct

SEQ ID NO: 190 - DNA, 30 bp, Human papillomavirus type 56
taactgaccctgcatttcttgatagacctg

SEQ ID NO: 191 - DNA, 30 bp, Human papillomavirus type 56
aactgaccctgcatttcttgatagacctgc

SEQ ID NO: 192 - DNA, 30 bp, Human papillomavirus type 56
gcatttcttgatagacctgcaacattagta

SEQ ID NO: 193 - DNA, 30 bp, Human papillomavirus type 56
catttcttgatagacctgcaacattagtat

SEQ ID NO: 194 - DNA, 30 bp, Human papillomavirus type 56
tttcttgatagacctgcaacattagtatct

SEQ ID NO: 195 - DNA, 30 bp, Human papillomavirus type 56
ttcttgatagacctgcaacattagtatctg

SEQ ID NO: 196 - DNA, 30 bp, Human papillomavirus type 56
gtactgacacatctttagctttttctccgt

SEQ ID NO: 197 - DNA, 30 bp, Human papillomavirus type 56
ctgacacatctttagctttttctccgtcgg

SEQ ID NO: 198 - DNA, 30 bp, Human papillomavirus type 56
tgacacatctttagctttttctccgtcggg

SEQ ID NO: 199 - DNA, 30 bp, Human papillomavirus type 56
gacacatctttagctttttctccgtcgggt

SEQ ID NO: 200 - DNA, 24 bp, Human papillomavirus type 58
gtgtaacctgtaacaacgccatga

SEQ ID NO: 201 - DNA, 24 bp, Human papillomavirus type 58
taggggctaaagtacattactacc

SEQ ID NO: 202 - DNA, 24 bp, Human papillomavirus type 58
aggggctaaagtacattactacca

SEQ ID NO: 203 - DNA, 24 bp, Human papillomavirus type 58
ctgtctacatccgtttcactgctt

SEQ ID NO: 204 - DNA, 24 bp, Human papillomavirus type 58
aactgtctacatccgtttcactgc

SEQ ID NO: 205 - DNA, 24 bp, Human papillomavirus type 58
acttttttattgttattgggactt

SEQ ID NO: 206 - DNA, 24 bp, Human papillomavirus type 58
attgttattgggacttttgatgga

SEQ ID NO: 207 - DNA, 41 bp, Human papillomavirus type 58
aggtagaagcggtaatagaacgaagaacaggagataatatt

SEQ ID NO: 208 - DNA, 30 bp, Human papillomavirus type 58
gaagcggtaatagaacgaagaacaggagat

SEQ ID NO: 209 - DNA, 40 bp, Human papillomavirus type 58
tcagatgtaagcagtgaaacggatgtagacagttgtaata

SEQ ID NO: 210 - DNA, 40 bp, Human papillomavirus type 58
cagatgtaagcagtgaaacggatgtagacagttgtaatac

SEQ ID NO: 211 - DNA, 30 bp, Human papillomavirus type 58
agatgtaagcagtgaaacggatgtagacag

SEQ ID NO: 212 - DNA, 40 bp, Human papillomavirus type 58
agatgtaagcagtgaaacggatgtagacagttgtaatact

SEQ ID NO: 213 - DNA, 30 bp, Human papillomavirus type 58
gatgtaagcagtgaaacggatgtagacagt

SEQ ID NO: 214 - DNA, 40 bp, Human papillomavirus type 58
gatgtaagcagtgaaacggatgtagacagttgtaatactg

SEQ ID NO: 215 - DNA, 30 bp, Human papillomavirus type 58
atgtaagcagtgaaacggatgtagacagtt

SEQ ID NO: 216 - DNA, 30 bp, Human papillomavirus type 58
tgtaagcagtgaaacggatgtagacagttg

SEQ ID NO: 217 - DNA, 30 bp, Human papillomavirus type 58
gtaagcagtgaaacggatgtagacagttgt

SEQ ID NO: 218 - DNA, 30 bp, Human papillomavirus type 58
taagcagtgaaacggatgtagacagttgta

SEQ ID NO: 219 - DNA, 30 bp, Human papillomavirus type 58
aagcagtgaaacggatgtagacagttgtaa

SEQ ID NO: 220 - DNA, 30 bp, Human papillomavirus type 58
agcagtgaaacggatgtagacagttgtaat

SEQ ID NO: 221 - DNA, 30 bp, Human papillomavirus type 58
ttatgctgacgatgctgatactatacatga

SEQ ID NO: 222 - DNA, 41 bp, Human papillomavirus type 58
tttgacactcctcttgtgtcattggaacctggtccagacat

SEQ ID NO: 223 - DNA, 36 bp, Human papillomavirus type 58
cactcctcttgtgtcattggaacctggtccagacat

SEQ ID NO: 224 - DNA, 30 bp, Human papillomavirus type 58
acactcctcttgtgtcattggaacctggtc

SEQ ID NO: 225 - DNA, 30 bp, Human papillomavirus type 58
cactcctcttgtgtcattggaacctggtcc

SEQ ID NO: 226 - DNA, 30 bp, Human papillomavirus type 58
acttgtggcaccacggttcgtttgtgtatc

SEQ ID NO: 227 - DNA, 30 bp, Human papillomavirus type 58
cttgtggcaccacggttcgtttgtgtatca

SEQ ID NO: 228 - DNA, 30 bp, Human papillomavirus type 58
atgctcagaaagtgctgtagaggactgtgt

SEQ ID NO: 229 - DNA, 30 bp, Human papillomavirus type 58
agtccatttattcctatatctccactaact

SEQ ID NO: 230 - DNA, 30 bp, Human papillomavirus type 58
tccatttattcctatatctccactaactcc

SEQ ID NO: 231 - DNA, 24 bp, Human papillomavirus type 59
ctgactttttaacacgtccatcca

SEQ ID NO: 232 - DNA, 24 bp, Human papillomavirus type 59
aacatccagacgcagcactgtaag

SEQ ID NO: 233 - DNA, 24 bp, Human papillomavirus type 59
tgtccctttattgtttctttgtcc

SEQ ID NO: 234 - DNA, 24 bp, Human papillomavirus type 59
gtccctttattgtttctttgtcct

SEQ ID NO: 235 - DNA, 30 bp, Human papillomavirus type 59
tggtaaagggtgtagtagaataagtgggtt

SEQ ID NO: 236 - DNA, 24 bp, Human papillomavirus type 59
actgtatggtggtaaagggtgtag

SEQ ID NO: 237 - DNA, 24 bp, Human papillomavirus type 59
ttcttggattgcacagtagttttg

SEQ ID NO: 238 - DNA, 24 bp, Human papillomavirus type 59
cattcttggattgcacagtagttt

SEQ ID NO: 239 - DNA, 41 bp, Human papillomavirus type 59
catgatataagccctataccacatgctgaagatattgaatt

SEQ ID NO: 240 - DNA, 36 bp, Human papillomavirus type 59
tataagccctataccacatgctgaagatattgaatt

SEQ ID NO: 241 - DNA, 30 bp, Human papillmoavirus type 59
tataagccctataccacatgctgaagatat

SEQ ID NO: 242 - DNA, 30 bp, Human papillomavirus type 59
ataagccctataccacatgctgaagatatt

SEQ ID NO: 243 - DNA, 30 bp, Human papillomavirus type 59
taagccctataccacatgctgaagatattg

SEQ ID NO: 244 - DNA, 30 bp, Human papillomavirus type 59
agccctataccacatgctgaagatattgaa

SEQ ID NO: 245 - DNA, 30 bp, Human papillomavirus type 59
gccctataccacatgctgaagatattgaat

SEQ ID NO: 246 - DNA, 41 bp, Human papillomavirus type 59
aatccccatcttgtttcctcctacacgcctagactactaac

SEQ ID NO: 247 - DNA, 36 bp, Human papillomavirus type 59
aatccccatcttgtttcctcctacacgcctagacta

SEQ ID NO: 248 - DNA, 30 bp, Human papillomavirus type 59
ccatcttgtttcctcctacacgcctagact

SEQ ID NO: 249 - DNA, 30 bp, Human papillomavirus type 59
tatgcagatattacagatgaagcacctact

SEQ ID NO: 250 - DNA, 30 bp, Human papillomavirus type 59
atgcagatattacagatgaagcacctacta

SEQ ID NO: 251 - DNA, 30 bp, Human papillomavirus type 59
tgcagatattacagatgaagcacctactag

SEQ ID NO: 252 - DNA, 30 bp, Human papillomavirus type 59
aacacaacttacaaacgccaaatagttagt

SEQ ID NO: 253 - DNA, 30 bp, Human papillomavirus type 59
cacaacttacaaacgccaaatagttagtca

SEQ ID NO: 254 - DNA, 30 bp, Human papillomavirus type 59
acaacttacaaacgccaaatagttagtcat

SEQ ID NO: 255 - DNA, 24 bp, Human papillomavirus type 66
gtagtatccttgggcagtgtgtgt

SEQ ID NO: 256 - DNA, 24 bp, Human papillomavirus type 66
gtatccttgggcagtgtgtgtcag

SEQ ID NO: 257 - DNA, 24 bp, Human papillomavirus type 66
tagttggcacagaaatacaggtga

SEQ ID NO: 258 - DNA, 30 bp, Human papillomavirus type 66
tagttggcacagaaatacaggtgagtaata

SEQ ID NO: 259 - DNA, 41 bp, Human papillomavirus type 66
agtaacacaccaaactccattttagtgctgtacgccatttt

SEQ ID NO: 260 - DNA, 33 bp, Human papillomavirus type 66
aacacaccaaactccattttagtgctgtacgcc

SEQ ID NO: 261 - DNA, 30 bp, Human papillomavirus type 66
aacacaccaaactccattttagtgctgtac

SEQ ID NO: 262 - DNA, 30 bp, Human papillomavirus type 66
acacaccaaactccattttagtgctgtacg

SEQ ID NO: 263 - DNA, 30 bp, Human papillomavirus type 66
cacaccaaactccattttagtgctgtacgc

SEQ ID NO: 264 - DNA, 30 bp, Human papillomavirus type 66
acaccaaactccattttagtgctgtacgcc

SEQ ID NO: 265 - DNA, 30 bp, Human papillomavirus type 66
aattcggttgcctagccttttgtccttatt

SEQ ID NO: 266 - DNA, 30 bp, Human papillomavirus type 66
attcggttgcctagccttttgtccttattt

SEQ ID NO: 267 - DNA, 30 bp, Human papillomavirus type 66
ttcggttgcctagccttttgtccttattta

SEQ ID NO: 268 - DNA, 24 bp, Human papillomavirus type 68
acagcacaggtacttttgaatatg

SEQ ID NO: 269 - DNA, 24 bp, Human papillomavirus type 68
agacagcacaggtacttttgaata

SEQ ID NO: 270 - DNA, 24 bp, Human papillomavirus type 68
atgcacctgatactgacaatacta

SEQ ID NO: 271 - DNA, 24 bp, Human papillomavirus type 68
acattgtccactactacagactct

SEQ ID NO: 272 - DNA, 24 bp, Human papillomavirus type 68
acattgcagcctttttattgttac

SEQ ID NO: 273 - DNA, 24 bp, Human papillomavirus type 68
aataacctagatgtaccagcatag

SEQ ID NO: 274 - DNA, 24 bp, Human papillomavirus type 68
gttaataacctagatgtaccagca

SEQ ID NO: 275 - DNA, 30 bp, Human papillomavirus type 68
caagacatataacaattattttgacacacg

SEQ ID NO: 276 - DNA, 30 bp, Human papillomavirus type 68
gcaggacattggacactacattgcatgacg

SEQ ID NO: 277 - DNA, 30 bp, Human papillomavirus type 68; identical to SEQ ID NO: 276

SEQ ID NO: 278 - DNA, 30 bp, Human papillomavirus type 68
ggacattggacactacattgcatgacgtta

SEQ ID NO: 279 - DNA, 30 bp, Human papillomavirus type 68; identical to SEQ ID NO: 278

SEQ ID NO: 280 - DNA, 48 bp, Human papillomavirus type 68
atagaaagcagtcctttagcaaagtcgccattacaggaattatcacta

SEQ ID NO: 281 - DNA, 37 bp, Human papillomavirus type 68
aagcagtcctttagcaaagtcgccattacaggaatta

SEQ ID NO: 282 - DNA, 30 bp, Human papillomavirus type 68
aagcagtcctttagcaaagtcgccattaca

SEQ ID NO: 283 - DNA, 30 bp, Human papillomavirus type 68
cctttagcaaagtcgccattacaggaatta

SEQ ID NO: 284 - DNA, 30 bp, Human papillomavirus type 68; identical to SEQ ID NO: 283

SEQ ID NO: 285 - DNA, 30 bp, Human papillomavirus type 68
ctttagcaaagtcgccattacaggaattat

SEQ ID NO: 286 - DNA, 30 bp, Human papillomavirus type 68; identical to SEQ ID NO: 285

SEQ ID NO: 287 - DNA, 30 bp, Human papillomavirus type 68
catttactactcgttcccacatatcagttc

SEQ ID NO: 288 - DNA, 30 bp, Human papillomavirus type 68
atttactactcgttcccacatatcagttcc

SEQ ID NO: 289 - DNA, 30 bp, Human papillomavirus type 68
cagttccttcattggcttctgctgcatcca

SEQ ID NO: 290 - DNA, 30 bp, Human papillomavirus type 68
agttccttcattggcttctgctgcatccac

SEQ ID NO: 291 - DNA, 30 bp, Human papillomavirus type 68
ttccttcattggcttctgctgcatccacta

SEQ ID NO: 292 - DNA, 30 bp, Human papillomavirus type 68
tccttcattggcttctgctgcatccactac

SEQ ID NO: 293 - DNA, 30 bp, Human papillomavirus type 68
cttcattggcttctgctgcatccactacat

SEQ ID NO: 294 - DNA, 30 bp, Human papillomavirus type 68
ttcattggcttctgctgcatccactacata

SEQ ID NO: 295 - DNA, 30 bp, Human papillomavirus type 68
tggcttctgctgcatccactacatatacta

SEQ ID NO: 296 - DNA, 41 bp, Human papillomavirus type 68
tagatacataccgctacctacaatcagcagcaattacatgt

SEQ ID NO: 297 - DNA, 41 bp, Human papillomavirus type 68; identical to SEQ ID NO: 296

SEQ ID NO: 298 - DNA, 30 bp, Human papillomavirus type 68
acataccgctacctacaatcagcagcaatt

SEQ ID NO: 299 - DNA, 30 bp, Human papillomavirus type 68
aaaaggacgcccctgcacctgttaaaaaag

SEQ ID NO: 300 - DNA, 30 bp, Human papillomavirus type 68
tatatgaatttgcctttagtgacctatgtg

SEQ ID NO: 301 - DNA, 30 bp, Human papillomavirus type 68; identical to SEQ ID NO: 300

SEQ ID NO: 302 - DNA, 30 bp, Human papillomavirus type 68
atgaatttgcctttagtgacctatgtgtag

SEQ ID NO: 303 - DNA, 30 bp, Human papillomavirus type 68; identical to SEQ ID NO: 302

SEQ ID NO: 304 - DNA, 30 bp, Human papillomavirus type 68
aagtggaaactaactcggaggtaactgtag

SEQ ID NO: 305 - DNA, 30 bp, Human papillomavirus type 68
gatcctaaatcacctactacccaacttaaa

SEQ ID NO: 306 - DNA, 30 bp, Human papillomavirus type 68; identical to SEQ ID NO: 305

SEQ ID NO: 307 - DNA, 30 bp, Human papillomavirus type 68
cctaaatcacctactacccaacttaaagta

SEQ ID NO: 308 - DNA, 30 bp, Human papillomavirus type 68; identical to SEQ ID NO: 307

SEQ ID NO: 309 - DNA, 30 bp, Human papillomavirus type 68; identical to SEQ ID NO: 293

SEQ ID NO: 310 - DNA, 30 bp, Human papillomavirus type 68; identical to SEQ ID NO: 294

SEQ ID NO: 311 - DNA, 30 bp, Human papillomavirus type 68
ccacagttgcctttaacaccctctactcca

SEQ ID NO: 312 - DNA, 30 bp, Human papillomavirus type 68
acagttgcctttaacaccctctactccaat

SEQ ID NO: 313 - DNA, 30 bp, Human papillomavirus type 68
cagttgcctttaacaccctctactccaatt

SEQ ID NO: 314 - DNA, 65 bp, Artificial Sequence
  a synthetic oligonucleotide
acatttgatcctcgtagtgatgttcctgataaaaaaaaaaaaaaaaaaaaaaaaaaaaaaaaaaa

SEQ ID NO: 315 - DNA, 65 bp, Artificial Sequence
  a synthetic oligonucleotide
tatgcgtgaccagctaccagaaagacgggcaaaaaaaaaaaaaaaaaaaaaaaaaaaaaaaaaaa

SEQ ID NO: 316 - DNA, 65 bp, Artificial Sequence
  a synthetic oligonucleotide
tatgcagatattacagatgaagcacctactaaaaaaaaaaaaaaaaaaaaaaaaaaaaaaaaaaa

(19) United States
(12) Patent Application Publication
(10) Pub. No.: US 2010/0304360 A1
(43) Pub. Date: Dec. 2, 2010

(54) GOLD NANOPARTICLE HPV GENOTYPING SYSTEM AND ASSAY

(75) Inventors: Peter Riccelli, Tinley Park, IL (US); Feng Sun, Deerfield, IL (US)

Correspondence Address: GREGORY T. PLETTA;Nanosphere, Inc., 4088 Commerical Avenue, Northbrook, IL, 60062-1829, US

(73) Assignee: Nanosphere, Inc., Northbrook, IL (US)

(21) Appl. No.: 12/642,395

(22) Filed: Dec. 18, 2009

Related U.S. Application Data
(60) Provisional application No. 61/138,942, filed on Dec. 18, 2008.

(51) Int. Cl. C12Q 1/70; C07H 21/00

(52) U.S. Cl. 435/5; 536/23.1

(57) ABSTRACT

The invention provides oligonucleotides, kits and methods for genotyping HPV.

### CROSS-REFERENCE TO RELATED APPLICATIONS

This application claims the benefit of the filing date of U.S. application Ser. No. 61/138,942, filed on Dec. 18, 2008, the disclosure of which is incorporated by reference herein.

### BACKGROUND

Cervical cancer is the second most common cancer in women worldwide and the seventh most common cause of cancer deaths in women in Europe. In low- and medium-resourced countries in Asia, Africa and Latin America, cervical cancer is the major cause of mortality and premature death among women in their most productive years. Cervical cytology screening has reduced cervical cancer morbidity and mortality but has significant shortcomings in terms of sensitivity and specificity. Infection with distinct types of human papillomavirus (HPV) is the primary etiologic factor in cervical carcinogenesis. This causal relationship has been exploited for the development of molecular technologies for viral detection to overcome limitations linked to cytologic cervical screening. HPV testing for high-risk types of HPV has been suggested for primary screening, triage of equivocal Pap smears or low-grade lesions and follow-up after treatment for cervical intraepithelial neoplasia (CIN). Determination of HPV genotype, viral load, integration status and RNA expression could further improve the effectiveness of HPV-based screening and triage strategies.

HPV testing detects almost all high-grade CINs identified by cytology (Cuzick et al., 2006; Cuzick et al., 2008). As a result, almost the same sensitivity is obtained with HPV testing alone as with both cytology and HPV testing together as primary screening tests if only HPV-positive or also HPV-negative women with abnormal cytology are referred to colposcopy. However, with the combined strategy, referrals to colposcopy are much more frequent and the probability that test-positive women actually have a high-grade CIN (the Positive Predictive Value, PPV) is substantially lower (Ronco et al., J. Natl. Cancer Inst., 98:765 (2006): Ronco et al., Lancet Oncol., 7:547 (2006)).

Other strategies to improve HPV testing include viral load, genotyping, testing for the RNA of the viral oncogenes E6 and E7 and testing for the over-expression of the p16-INK4A protein (Cuzick et al., Vaccine, 24:S90 (2006)).

### SUMMARY OF THE INVENTION

The invention relates to a genotyping assay and kit for diagnosing patients infected with high-risk (HR) human papillomavirus (HPV). Also provided is a method for detecting and genotyping specimen DNA in a manner that incorporates a control for clinical relevance. The invention provides isolated oligonucleotides for specifically amplifying HR-HPV DNA, e.g., by the polymerase chain reaction (PCR), and for detecting subtype-specific HR HPV. A kit of the invention includes at least one subtype-specific capture probe, at least one subtype-specific mediator probe, DNA-modified particles (DNA-P) such as gold nanoparticles (DNA-GNP) or silver nanoparticles (DNA-AgNP), or combinations thereof. Capture probes of the invention include a first nucleic acid sequence capable of hybridization to a first HPV-specific nucleic acid sequence or to a first HPV subtype-specific nucleic acid sequence. Mediator probes of the invention include a second nucleic acid sequence capable of hybridization to a second HPV-specific nucleic acid sequence or to a second HPV subtype-specific nucleic acid sequence, wherein the second nucleic acid sequence of the mediator probe hybridizes to a different HPV nucleic acid sequence relative to the capture probe. DNA-P include oligonucleotides capable of hybridization to a sequence contained in the mediator probe that is not HPV-specific, e.g., polydA or polyT.

In one embodiment, the invention provides a method for detecting high risk HPV in a sample. The method includes providing a substrate having a capture probe bound thereto, wherein at least a portion of the capture probe has a nucleic acid sequence that is complementary to at least a first portion of the genome of a HPV and providing a mediator probe, wherein at least a portion of the mediator probe has a nucleic acid sequence that is complementary to at least a second portion of the HPV genome that is different than the first portion and a nucleotide sequence that is complementary to a non-HPV sequence on oligonucleotides bound to a gold particle, wherein the nucleic acid sequence in the capture probe or the mediator probe, or both, are HPV-subtype specific. A sample suspected of having HPV that is optionally subjected to an amplification reaction with HPV-specific primers, is contacted with the substrate, the mediator probe and gold particles having oligonucleotides with sequences that are complementary to the nucleotide sequence in the mediator probe under conditions that are effective for the hybridization of the nucleic acid sequence in the capture probe and the nucleic acid sequence in the mediator probe to amplified HPV DNA in the sample and for the hybridization of the nucleotide sequence in the mediator probe to the oligonucleotides bound to the gold particle. The substrate is washed to remove non-specifically bound material and it is determined whether gold particles are bound to the substrate. The presence of bound particles is indicative of the presence of a specific subtype of HPV in the sample.

In one embodiment, a capture probe has about 25 to 55 nucleotides of HPV-specific sequence. In one embodiment, a capture probe includes a nucleotide sequence corresponding to one of SEQ ID No. 8-22, 38-50, 63-70, 83-94, 103-111, 119-130, 144-154, 168-175, 186-195, 207-225, 239-248, 259-264, or 276-299, a sequence with at least 80% sequence identity thereto, or the complement thereof. In one embodiment, a capture probe includes a nucleotide sequence corresponding to one of SEQ ID No. 8-22, 38-50, 63-70, 83-94, 103-111, 119-130, 144-154, 168-175, 186-195, 207-225, 239-248, 259-264, or 276-299, a sequence with at least 90% sequence identity thereto, or the complement thereof. In one embodiment, a capture probe includes a sequence corresponding one of SEQ ID No. 23-31, 51-55, 71-78, 95-98, 112-115, 131-135, 155-161, 176-181, 195-199, 226-230, 249-254, 264-267, or 300-313, or a sequence with at least 80% sequence identity thereto, or the complement thereof. In one embodiment, a capture probe includes a sequence corresponding one of SEQ ID No. 23-31, 51-55, 71-78, 95-98, 112-115, 131-135, 155-161, 176-181, 195-199, 226-230, 249-254, 264-267, or 300-313, or a sequence with at least 90% sequence identity thereto, or the complement thereof. A capture probe may include other sequences so long as they do not substantially decrease hybridization efficiency of the probe to a target HPV sequence, e.g., the other sequences are 5′ and/or 3′ to the sequences that specifically hybridize to HPV sequences, for example, the other sequence may be a tag sequence or a barcode sequence.

In one embodiment, a mediator probe has about 25 to 55 nucleotides of HPV-specific sequence. In one embodiment, a mediator probe includes a nucleotide sequence corresponding to one of SEQ ID No. 8-22, 38-50, 63-70, 83-94, 103-111, 119-130, 144-154, 168-175, 186-195, 207-225, 239-248, 259-264, or 276-299, a sequence with at least 80% sequence identity thereto, or the complement thereof. In one embodiment, a mediator probe includes a nucleotide sequence corresponding to one of SEQ ID No. 8-22, 38-50, 63-70, 83-94, 103-111, 119-130, 144-154, 168-175, 186-195, 207-225, 239-248, 259-264, or 276-299, a sequence with at least 90% sequence identity thereto, or the complement thereof. In one embodiment, a mediator probe includes a sequence corresponding one of SEQ ID No. 23-31, 51-55, 71-78, 95-98, 112-115, 131-135, 155-161, 176-181, 195-199, 226-230, 249-254, 264-267, or 300-313, a sequence with at least 80% sequence identity thereto, or the complement thereof. In one embodiment, a mediator probe includes a sequence corresponding one of SEQ ID No. 23-31, 51-55, 71-78, 95-98, 112-115, 131-135, 155-161, 176-181, 195-199, 226-230, 249-254, 264-267, or 300-313, or a sequence with at least 90% sequence identity thereto, or the complement thereof. A mediator probe also includes a sequence complementary to sequences on oligonucleotides attached to a particle, and may include other sequences so long as they do not substantially decrease hybridization efficiency of the probe to a target HPV sequence and the oligonucleotide, e.g., the other sequences 5′ and/or 3′ to the sequences that specifically hybridize to HPV sequences, for example, the other sequence may be a tag sequence or a barcode sequence.

In one embodiment, capture probes and mediator probes useful in the methods of the invention include HPV-specific sequences that do not overlap and do not cross-hybridize, e.g., do not compete for binding to the same target nucleotide sequence. In one embodiment, a selected capture and mediator probe pair hybridize to their respective target sequences under the same stringency conditions. In one embodiment, a selected capture probe and mediator probe that hybridize under different stringency conditions may be employed, e.g., the probe that hybridizes and/or remains hybridized under both hybridization conditions is hybridized to the target first. In one embodiment, a capture probe useful in the methods of the invention has sequences that hybridize to HPV sequences that are about 50 to about 2000 nucleotides apart from sequences to which the mediator probe hybridizes. n one embodiment, a capture probe useful in the methods of the invention has sequences that hybridize to HPV sequences that are about 1 to about 50 nucleotides apart from sequences to which the mediator probe hybridizes. In one embodiment, a capture probe useful in the methods of the invention has sequences that hybridize to HPV sequences that are about 500 to about 1000 nucleotides apart from sequences to which the mediator probe hybridizes.

The invention also provides a kit. For example, in one embodiment, the kit may include at least one HPV subtype-specific capture probe and optionally at least one HPV subtype-specific mediator probe, and/or DNA-P. In one embodiment the kit may include at least one subtype-specific mediator probe and optionally at least one subtype-specific capture probe, and/or DNA-P. In one embodiment, the kit includes a DNA control that may be co-amplified with a clinical sample in order to provide a clinically relevant cutoff point for detection of the HR-HPV virus. In one embodiment, the kit also includes at least one primer pair for HPV subtype-specific amplification of viral DNA in a sample.

Also provided are isolated oligonucleotides which include one of SEQ ID Nos. 1-313, a sequence with at least 80% sequence identity thereto, or the complement thereof, or a fragment thereof with at least 10, e.g., at least 15 or 20, contiguous nucleotides, of one of SEQ ID Nos. 1-313, a sequence with at least 80% sequence identity thereto, or the complement thereof. The HPV-specific sequences in the isolated oligonucleotides may be useful as primers, e.g., amplification primers, or probes.

### BRIEF DESCRIPTION OF THE FIGURES

FIGS. 1A-F. Verigene gold nanoparticle slide data (in duplicate) of multiplexed PCR samples with varying input copy number of plasmids. The capture probes are spotted in triplicate and highlighted by colored circles. The average median of the intensities is shown above the slide data.

FIG. 2. Verigene gold nanoparticle slide data of multiplexed PCR clinical samples with 250 input copies of control plasmid. The capture probes are spotted in triplicate and highlighted by colored circles. The average median of the intensities is shown above the slide data.

FIGS. 3A-Z and 3AA. Graphs showing the specificity of capture and mediator probes of the invention in detecting subtypes of HPV.

### DETAILED DESCRIPTION OF THE INVENTION

### Definitions

A “nucleotide” is a subunit of a nucleic acid comprising a purine or pyrimidine base group, a 5-carbon sugar and a phosphate group. The 5-carbon sugar found in RNA is ribose. In DNA, the 5-carbon sugar is 2′-deoxyribose. The term also includes analogs of such subunits, such as a methoxy group (MeO) at the 2′ position of ribose.

A “biological sample” can be obtained from an organism, e.g., it can be a physiological fluid or tissue sample, such as one from a human patient, a laboratory mammal such as a mouse, rat, pig, monkey or other member of the primate family.

“Tₘ” refers to the temperature at which 50% of the duplex is converted from the hybridized to the unhybridized form.

One skilled in the art will understand that the oligonucleotides useful in the methods can vary in sequence. For instance, amplification primers useful to amplify either HPV-specific nucleic acid sequences, e.g., sequences that are not specific for one or are specific for a few different HPV subtypes, or HPV subtype-specific nucleic acid sequences, may have less than 100% sequence identity to the HPV genomic sequences in the biological sample due to the presence of at least one mismatch. In one embodiment, an amplification primer useful to amplify either HPV-specific sequences or HPV-subtype specific nucleic acid sequences, may have less than 100% sequence identity to the amplification primers disclosed herein, for instance, SEQ ID No. 1-7, 32-37, 79-82, 96-102, 116-118, 136-143, 162-167, 182-185, 200-206, 231-238, 255-258, or 268-275, or a fragment thereof. In one embodiment, capture probe sequences may include either HPV-specific sequences or HPV-subtype specific nucleic acid sequences, that have less than 100% sequence identity to the HPV genomic sequences in the biological sample (and thus to amplified HPV sequences) due to the presence of at least one mismatch. In one embodiment, capture probe sequences may have less than 100% sequence identity to the capture probe sequences disclosed herein, e.g., one of SEQ ID No. 8-22, 38-50, 63-70, 83-94, 103-111, 119-130, 144-154, 168-175, 186-195, 207-225, 239-248, 259-264, or 276-299, the complement thereof, or a fragment thereof. In one embodiment, mediator probe sequences may include either HPV-specific sequences or HPV-subtype specific nucleic acid sequences, that have less than 100% sequence identity to the HPV genomic sequences in the biological sample (and thus to amplified HPV sequences) due to the presence of at least one mismatch. In one embodiment, mediator probe sequences may have less than 100% sequence identity to the capture probe sequences disclose herein, e.g., one of SEQ ID No. 23-31, 51-55, 71-78, 95-98, 112-115, 131-135, 155-161, 176-181, 195-199, 226-230, 249-254, 264-267, or 300-313, the complement thereof, or a fragment thereof. Thus, the percentage of identical bases or the percentage of perfectly complementary bases between oligonucleotides and sequence the oligonucleotides hybridize to may be less than 100% but in the region of complementarity have at least 80%, 85%, 90%, 95%, 98%, or 99% identity. The oligonucleotides may also contain sequences that have no complementarity, however, the sequences that do not have complementarity do not prevent the hybridization of the complementary sequences.

By “sufficiently complementary” or “substantially complementary” is meant nucleic acids having a sufficient amount of contiguous complementary nucleotides to form a hybrid that is stable.

“RNA and DNA equivalents” refer to RNA and DNA molecules having the same complementary base pair hybridization properties. RNA and DNA equivalents have different sugar groups (i.e., ribose versus deoxyribose), and may differ by the presence of uracil in RNA and thymine in DNA. The difference between RNA and DNA equivalents do not contribute to differences in substantially corresponding nucleic acid sequences because the equivalents have the same degree of complementarity to a particular sequence.

### Methods and Kits

The invention provides a method for detecting and genotyping HR-HPV from a sample containing HPV DNA while also optionally determining if the HPV infection is clinically relevant. In one embodiment, the assay is based around first isolating the HPV DNA from a clinical sample and then amplifying the HPV DNA by a multiple PCR using HPV subtype-specific primers. In one embodiment, the amplified DNA is then hybridized with a HPV subtype-specific capture probe oligonucleotide bound to the solid support, followed by hybridization with a HPV subtype-specific mediator probe that contain 3′-tails comprising a run of about 10 to about 50, e.g., about 20 to about 35, adenosine phosphates (polyA). In one embodiment, this is followed by hybridization with DNA-GNP with attached 20mer dT oligonucleotides. The gold nanoparticles may be detected by catalytically reducing silver onto the surface of the particle, followed by imaging of the silver by detection of light scattered from the silver enhanced gold nanoparticles. Incorporating a DNA control at a specific copy number into the sample allows for co-amplification in a multiplex PCR and normalizes the readout intensity values to a predefined clinically relevant threshold.

### Oligonucleotides

Each oligonucleotide sequence of the invention including those in primers, capture probes, mediator probes or attached to particles, has the ability to hybridize to at least one other specific nucleotide sequence that is HPV-specific, HPV-subtype specific, or non-HPV specific having a sequence sufficiently complementary.

Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are contemplated for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

The term “oligonucleotide” as used herein includes modified forms as discussed herein as well as those otherwise known in the art which are used to regulate gene expression Likewise, the term “nucleotides” as used herein is interchangeable with modified forms as discussed herein and otherwise known in the art. In certain instances, the art uses the term “nucleobase” which embraces naturally-occurring nucleotides as well as modifications of nucleotides that can be polymerized. Herein, the terms “nucleotides” and “nucleobases” are used interchangeably to embrace the same scope unless otherwise noted.

In various aspects, methods include oligonucleotides which are DNA oligonucleotides, RNA oligonucleotides, or combinations of the two types. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function as a probe in hybridization, as a primer in PCR and DNA sequencing. Examples of universal bases include but are not limited to 5′-nitroindole-2′-deoxyriboside, 3-nitropyrrole, inosine and pypoxanthine.

Modified Backbones. Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

Modified Sugar and Internucleoside Linkages. In still other embodiments, oligonucleotide mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligo consists of 2 to 4, desirably 3, groups/atoms selected from —CH₂—, —O—, —S—, —NR^{II}—, C═O, C═NR^{II}, >C═S, —Si(R″)₂—, —SO—, —S(O)₂—, —P(O)₂—, —PO(BH₃)—, —P(O,S)—,—P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH=(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N=(including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH=(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. Patent Publication No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples herein below.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Natural and Modified Bases. Oligonucleotides may also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tₘ differential of 15, 12, 10, 8, 6, 4, or 2° C. or less. Exemplary modified bases are described in EP 1 072 679 and WO 97/12896.

An oligonucleotide, or modified form thereof, may be from about 20 to about 100 nucleotides in length. It is also contemplated wherein the oligonucleotide is about 20 to about 90 nucleotides in length, about 20 to about 80 nucleotides in length, about 20 to about 70 nucleotides in length, about 20 to about 60 nucleotides in length, about 20 to about 50 nucleotides in length about 20 to about 45 nucleotides in length, about 20 to about 40 nucleotides in length, about 20 to about 35 nucleotides in length, about 20 to about 30 nucleotides in length, about 20 to about 25 nucleotides in length, or about 15 to about 90 nucleotides in length, about 15 to about 80 nucleotides in length, about 15 to about 70 nucleotides in length, about 15 to about 60 nucleotides in length, about 15 to about 50 nucleotides in length about 15 to about 45 nucleotides in length, about 15 to about 40 nucleotides in length, about 15 to about 35 nucleotides in length, about 15 to about 30 nucleotides in length, about 15 to about 25 nucleotides in length, or about 15 to about 20 nucleotides in length, and all oligonucleotides intermediate in length of the sizes specifically disclosed to the extent that the oligonucleotide is able to achieve the desired result. Accordingly, oligonucleotides of 15, 16, 17, 18, 19, 20, 21, 22, 23, 24,25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated. For example, a primer for amplification may be from about 20 to about 35, or any integer in between, nucleotides in length, and a probe may be from about 25 to about 55, or any integer in between, nucleotides in length

“Hybridization,” which is used interchangeably with the term “complex formation” herein, means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

In various aspects, the methods include use of oligonucleotides which are 100% complementary to another sequence, e.g., a sequence in HPV genomic DNA or another oligonucelotide sequence useful in the methods, i.e., a perfect match, while in other aspects, the individual oligonucleotides are at least (meaning greater than or equal to) about 95% complementary to all or part of another sequence, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to that sequence, so long as the oligonucleotide is capable of hybridizing to the target sequence.

It is understood in the art that the sequence of the oligonucleotide used in the methods need not be 100% complementary to a target sequence to be specifically hybridizable. Moreover, an oligonucleotide may hybridize to a target sequence over one or more segments such that intervening or adjacent segments are not involved in the hybridization event (e.g., a loop structure or hairpin structure). Percent complementarity between any given oligonucleotide and a target sequence can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The stability of the hybrids is chosen to be compatible with the assay conditions. This may be accomplished by designing the nucleotide sequences in such a way that the Tₘ will be appropriate for standard conditions to be employed in the assay. The position at which the mismatch occurs may be chosen to minimize the instability of hybrids. This may be accomplished by increasing the length of perfect complementarity on either side of the mismatch, as the longest stretch of perfectly homologous base sequence is ordinarily the primary determinant of hybrid stability. In one embodiment, the regions of complementarity may include G:C rich regions of homology. The length of the sequence may be a factor when selecting oligonucleotides for use with particles. In one embodiment, at least one of the oligonucleotides has 100 or fewer nucleotides, e.g., has 15 to 50, 20 to 40, 15 to 30, or any integer from 15 to 50, nucleotides. Oligonucleotides having extensive self-complementarity should be avoided. Less than 15 nucleotides may result in a oligonucleotide complex having a too low a melting temperature to be suitable in the disclosed methods. More than 100 nucleotides may result in a oligonucleotide complex having a too high melting temperature to be suitable in the disclosed methods. Thus, oligonucleotides are of about 15 to about 100 nucleotides, e.g., about 20 to about 70, about 22 to about 60, or about 25 to about 50 nucleotides in length.

### Particles

Particles for use in the methods or kits of the invention may be formed of any material that allows for detection and/or genotyping of HPV. In one embodiment, the particles are formed of a noble metal. In one embodiment, the particles are nanoparticles (NP). In general, nanoparticles (NPs) contemplated include any compound or substance with a high loading capacity for an oligonucleotide as described herein, including for example and without limitation, a metal, a semiconductor, and an insulator particle compositions, and a dendrimer (organic or inorganic). The term “functionalized nanoparticle,” as used herein, refers to a nanoparticle having at least a portion of its surface modified with an oligonucleotide. In one embodiment, the nanoparticles are gold or silver nanoparticles.

Thus, nanoparticles are contemplated for use in the methods which comprise a variety of inorganic materials including, but not limited to, metals, semi-conductor materials or ceramics as described in U.S. Patent Publication No. 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also contemplated for use in producing nanoparticles.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example., ferromagnetite) colloidal materials, as well as silica containing materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992).

In practice, methods are provided using any suitable nanoparticle having oligonucleotides attached thereto that are in general suitable for use in detection assays known in the art to the extent and do not interfere with oligonucleotide complex formation, i.e., hybridization to form a double-strand or triple-strand complex. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles, aggregate particles, isotropic (such as spherical particles) and anisotropic particles (such as non-spherical rods, tetrahedral, prisms) and core-shell particles such as the ones described in U.S. Pat. No. 7,238,472 and International Patent Publication No. WO 2002/096262, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preaparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

Suitable nanoparticles are also commercially available from, for example, Ted Pella, Inc. (gold), Amersham Corporation (gold) and Nanoprobes, Inc. (gold).

Also as described in U.S. Patent Publication No. 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pp. 16-47.

As further described in U.S. Patent Publication No. 20030147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

At least one oligonucleotide is bound to the nanoparticle through a 5′ linkage and/or the oligonucleotide is bound to the nanoparticle through a 3′ linkage. In various aspects, at least one oligonucleotide is bound through a spacer to the nanoparticle. In these aspects, the spacer is an organic moiety, a polymer, a water-soluble polymer, a nucleic acid, a polypeptide, and/or an oligosaccharide. Methods of functionalizing the oligonucleotides to attach to a surface of a nanoparticle are well known in the art. See Whitesides, Proceedings of the Robert A. Welch Foundation 39th Conference On Chemical Research Nanophase Chemistry, Houston, Tex., pages 109-121 (1995). See also, Mucic et al. Chem. Comm. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4:370-377 (1974) and Matteucci and Caruthers, J. Am. Chem. Soc., 103:3185-3191 (1981) for binding of oligonucleotides to silica and glass surfaces, and Grabaretal., Anal. Chem., 67:735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attach oligonucleotides to nanoparticles: Nuzzo et al., J. Am. Chem. Soc., 109:2358 (1987) (disulfides on gold); Allara and Nuzzo, Langmuir, 1:45 (1985) (carboxylic acids on aluminum); Allara and Tompkins, J. Colloid Interface Sci., 49:410-421 (1974) (carboxylic acids on copper); Iler, The Chemistry Of Silica, Chapter 6, (Wiley 1979) (carboxylic acids on silica); Timmons and Zisman, J. Phys. Chem., 69:984-990 (1965) (carboxylic acids on platinum); Soriaga and Hubbard, J. Am. Chem. Soc., 104:3937 (1982) (aromatic ring compounds on platinum); Hubbard, Acc. Chem. Res., 13:177 (1980) (sulfolanes, sulfoxides and other functionalized solvents on platinum); Hickman et al., J. Am. Chem. Soc., 111:7271 (1989) (isonitriles on platinum); Maoz and Sagiv, Langmuir, 3:1045 (1987) (silanes on silica); Maoz and Sagiv, Langmuir, 3:1034 (1987) (silanes on silica); Wasserman et al., Langmuir, 5:1074 (1989) (silanes on silica); Eltekova and Eltekov, Langmuir, 3:951 (1987) (aromatic carboxylic acids, aldehydes, alcohols and methoxy groups on titanium dioxide and silica); Lec et al., J. Phys. Chem., 92:2597 (1988) (rigid phosphates on metals).

### Nanoparticle Size

In various aspects, methods provided include those utilizing nanoparticles which range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or amount surface area that can be derivatized as described herein.

### Exemplary Solid Substrates

Any substrate which allows observation of a detectable change, e.g., an optical change, may be employed in the methods of the invention. Suitable substrates include transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO), silicon dioxide (SiO₂), silicon oxide (SiO), silicon nitride, etc.)). The substrate can be any shape or thickness, but generally is flat and thin. In one embodiment, the substrates are transparent substrates such as glass (e.g., glass slides) or plastics (e.g., wells of microtiter plates).

### Exemplary System

The Verigene System consists of three major components, a disposable cartridge with on board reagents for each assay (Verigene test cartridge, (see, e.g., U.S. Pat. No. 5,599,668)), an automated fluid processor (Verigene Processor) to execute the assay protocol, and the imaging device (Verigene reader) to read the assay result (see, e.g., U.S. Pat. No. 7,110,585).

The disposable cartridge comprises a glass microarray slide captured by a substrate holder; a silicone gasket between the slide and plastic housing, which forms one individual 12 μL reaction chamber; and a plastic housing. The housing contains multiple reagent wells for on board reagent storage that are covered by a snap-on cover. Routing of fluids from the reagent wells to the reaction chamber is accomplished by a microfluidic valve plate with predetermined fluid paths for each step of the reaction.

During an assay, reagents are pumped from the reagent wells through a microfluidic channel and into the reaction chamber. The temperature subsystem is composed of resistive heating and thermoelectric cooling elements that have the ability to control fluid temperature in the hybridization cartridge from about 15° C. up to about 60° C., within about 1° C. to 2° C. of accuracy.

Once a disposable cartridge is inserted into the Verigene processor, the instrument automatically reads the bar code and alerts the Verigene System of the required processing protocol to be followed. This action triggers the start of the automated assay. Reagents are processed sequentially based on time, temperature, and motion requirements specified in the assay protocol. Waste reagents are stored in the disposable cartridge.

One patient sample can be analyzed per disposable cartridge. Upon completion of the assay, the disposable cartridge is removed from the automated fluid processor and the top portion comprising the microfluidic channels and the hybridization chambers is removed from the substrate holder (see, e.g., U.S. Pat. No. 7,163,823). Removing the hybridization cartridge automatically empties waste into a sealed container that is disposed of with the reagent cartridge.

The substrate holder with the microarray slide is inserted into the Verigene reader, which automatically begins imaging and data analysis.

The invention will be further described by the following non-limiting examples.

### Example

Tables 1-13 provide sequence data for exemplary oligonucleotides useful to amplify and/or detect a HR-HPV of interest. Each table presents the oligonucleotide sequences for a specific HR HPV subtype and lists exemplary PCR primers for HPV subtype-specific amplification, exemplary sequences for oligonucleotide capture probes that may be bound to a solid support, and exemplary sequences for mediator probes. Note that the mediator probe sequences listed in the tables only show the HPV subtype-specific sequences. All of these mediator probes contain 3′-tails with about 20 to about 35 linked adenosine phosphate molecules (polyA).

Other primers for amplification, including those with nucleotide substitutions relative to those shown in the tables, may be employed in the methods and kits of the invention. Other capture probes and other mediators probes, including those with nucleotide substitutions relative to those shown in the tables, may be employed in the methods and kits of the invention. In addition, the sequence of a mediator probe shown in the tables may be employed as a capture probe and the sequence of a capture probe shown in the tables may be employed as a mediator probe so long as the mediator probe contains a sequence that binds to the oligonucleotide affixed to a detectable particle.

The high sensitivity of the Verigene system readily enables the use of a control DNA plasmid to determine the clinical relevance of a patient's HPV infection. By co-amplifying a known copy number of a control HPV sequence containing DNA plasmid, the multiplex PCR can be stopped during the exponential phase and detected on the Verigene platform. Since these reactions are stopped during the exponential phase of the PCR, before reaching the linear or plateau phases, the amplified HPV DNA can be quantified relative to the amplified control DNA. The Verigene system intensity value of the amplified control DNA can normalize the amplified HPV DNA and set a clinical relevance threshold. This normalizing method is facilitated by the high sensitivity of the Verigene System because it allows for stopping and detecting the PCR at cycles earlier than what would be possible with a fluorescent readout. When dealing with clinical samples of unknown DNA amounts, this greatly facilitates being able to stop the reaction in the exponential phase of the PCR.

TABLE 1
HPV primers and probes for amplifying and typing HPV type 16

| SEQ ID NO: | | Sequence (5′-3′) |
|---|---|---|
| | PCR primers | |
| 1 | hpv16_18779_3endtrunc | CGTAGACATTCGTACTTTGGAAGA |
| 2 | hpv16_18780_5endtrunc | ATTCGTACTTTGGAAGACCTGTTA |
| 3 | hpv16_22634_53endtrunc | TTACTACTTCAACTGATACCACAC |
| 4 | hpv16_22634_5endtrunc | CTACTTCAACTGATACCACACCTG |
| 5 | hpv16_19414_3endtrunc_rc | GTAAGTGGTGTTTGGCATATAGTG |
| 6 | hpv16_19414_5endtrunc_rc | ATATTTGTAAGTGGTGTTTGGCAT |
| 7 | hpv16_23344_rc | GGTTGTAGAAGTATCTGTAATAAAGTCATC |
| | Capture probes | |
| 8 | capv1_16_18107 | TTCAGGACCCACAGGAGCGACCCAGAAAGT |
| 9 | capv1_16_18110 | AGGACCCACAGGAGCGACCCAGAAAGTTAC |
| 10 | capv1_16_18112 | GACCCACAGGAGCGACCCAGAAAGTTACCA |
| 11 | capv1_16_18114 | CCCACAGGAGCGACCCAGAAAGTTACCACA |
| 12 | hpv16_cap_18973 | TGACGAGAACGAAAATGACAGTGATACAGGTGAAGATTTGG |
| 13 | hpv16_cap_18973_extend_1 | TGACGAGAACGAAAATGACAGTGATACAGGTGAAGATTTGG |
| 14 | capv1_16_18970 | ACGAGAACGAAAATGACAGTGATACAGGTG |
| 15 | capv1_16_18971 | CGAGAACGAAAATGACAGTGATACAGGTGA |
| 16 | capv1_16_18972 | GAGAACGAAAATGACAGTGATACAGGTGAA |
| 17 | capv1_16_18973 | AGAACGAAAATGACAGTGATACAGGTGAAG |
| 18 | capv1_16_18974 | GAACGAAAATGACAGTGATACAGGTGAAGA |
| 19 | capv1_16_18975 | AACGAAAATGACAGTGATACAGGTGAAGAT |
| 20 | hpv16_cap_22767 | ACTGGAGGGCATTTTACACTTTCATCATCCACTATTAGTAC |
| 21 | hpv16_cap_22767_extend_1 | ACTGGAGGGCATTTTACACTTTCATCATCCACTATTAGTAC |
| 22 | hpv16_cap_23109 | AGGCCAGCATTAACCTCTAGGCGTACTGGCATTAGGTACAG |
| | Mediator probes | |
| 23 | medv1_16_19232 | AGGAGATTATTTGAAAGCGAAGACAGCGGG |
| 24 | medv1_16_19233 | GGAGATTATTTGAAAGCGAAGACAGCGGGT |
| 25 | medv1_16_19234 | GAGATTATTTGAAAGCGAAGACAGCGGGTA |
| 26 | medv1_16_22953 | TGCTTTTGTAACCACTCCCACTAAACTTAT |
| 27 | medv1_16_22953 | TGCTTTTGTAACCACTCCCACTAAACTTAT |
| 28 | medv1_16_23263 | ATAACACCTTCTACATATACTACCACTTCA |
| 29 | medv1_16_24920 | AAACATACACCTCCAGCACCTAAAGAAGAT |
| 30 | medv1_16_24922 | ACATACACCTCCAGCACCTAAAGAAGATGA |
| 31 | medv1_16_24926 | ACACCTCCAGCACCTAAAGAAGATGATCCC |

TABLE 2
HPV primers and probes for amplifying and typing HPV type 18

| SEQ ID NO: | | Sequence (5′-3′) |
|---|---|---|
| | PCR primers | |
| 32 | hpv18_38408_3endtrunc | TATCACACCTTCGTCTACCTCTGT |
| 33 | hpv18_38471_3endtrunc | TGATCCGTCCATTATTGAAGTTCC |
| 34 | hpv18_38989_5endtrunc_rc | CATTGTCCTCCGTGGCAGATACTA |
| 35 | hpv18_38993_3endtrunc_rc | TTGTCCTCCGTGGCAGATACTAAA |
| 36 | hpv18_38989_5endtrunc_rc | CATTGTCCTCCGTGGCAGATACTA |
| 37 | hpv18_38993_3endtrunc_rc | TTGTCCTCCGTGGCAGATACTAAA |
| | Capture probes | |
| 38 | capv1_18_38686 | CAGTGGCTAACCCTGAGTTTCTTACACGTC |
| 39 | capv1_18_38687 | AGTGGCTAACCCTGAGTTTCTTACACGTCC |
| 40 | hpv18_cap_38693 | GTGGCTAACCCTGAGTTTCTTACACGTCCATCCTCTTTAAT |
| 41 | hpv18_cap_38693_extend_1 | GTGGCTAACCCTGAGTTTCTTACACGTCCATCCTCTTTAAT |
| 42 | capv1_18_38693 | TAACCCTGAGTTTCTTACACGTCCATCCTC |
| 43 | capv1_18_38694 | AACCCTGAGTTTCTTACACGTCCATCCTCT |
| 44 | capv1_18_38695 | ACCCTGAGTTTCTTACACGTCCATCCTCTT |
| 45 | capv1_18_38696 | CCCTGAGTTTCTTACACGTCCATCCTCTTT |
| 46 | capv1_18_38697 | CCTGAGTTTCTTACACGTCCATCCTCTTTA |
| 47 | capv1_18_38698 | CTGAGTTTCTTACACGTCCATCCTCTTTAA |
| 48 | capv1_18_38699 | TGAGTTTCTTACACGTCCATCCTCTTTAAT |
| 49 | capv1_18_38700 | GAGTTTCTTACACGTCCATCCTCTTTAATT |
| 50 | capv1_18_38772 | ACATTTGATCCTCGTAGTGATGTTCCTGAT |
| | Mediator probes | |
| 51 | medv1_18_38687 | AGTGGCTAACCCTGAGTTTCTTACACGTCC |
| 52 | medv1_18_38694 | AACCCTGAGTTTCTTACACGTCCATCCTCT |
| 53 | medv1_18_38696 | CCCTGAGTTTCTTACACGTCCATCCTCTTT |
| 54 | medv1_18_38699 | TGAGTTTCTTACACGTCCATCCTCTTTAAT |
| 55 | medv1_18_38772 | ACATTTGATCCTCGTAGTGATGTTCCTGAT |

TABLE 3
HPV primers and probes for amplifying and typing HPV type 31

| SEQ ID NO: | | Sequence (5′-3′) |
|---|---|---|
| | PCR primers | |
| 56 | hpv31_100787_53endtrunc | CAGACGTTATACCTAAAATAGAAC |
| 57 | hpv31_100788_53endtrunc | AGACGTTATACCTAAAATAGAACA |
| 58 | hpv31_103905_3endtrunc | CTATAATTTAGGTGTCACGCCATA |
| 59 | hpv31_103906_3endtrunc | TATAATTTAGGTGTCACGCCATAG |
| 60 | hpv31_101128_3endtrunc_rc | AACACTTGTTACATCTAAAATTGC |
| 61 | hpv31_104191_53endtrunc_rc | CACATAGTTGAACTACAGTTGTAT |
| 62 | hpv31_104192_53endtrunc_rc | ACACATAGTTGAACTACAGTTGTA |
| | Capture probes | |
| 63 | hpv31_cap_100917 | ATATGTCCCTCTTAGTACACGTCCTTCTACAGTATCTGAGG |
| 64 | hpv31_cap_100917_extend_1 | ATATGTCCCTCTTAGTACACGTCCTTCTACAGTATCTGAGG |
| 65 | capv1_31_100915 | TATGTCCCTCTTAGTACACGTCCTTCTACA |
| 66 | capv1_31_100916 | ATGTCCCTCTTAGTACACGTCCTTCTACAG |
| 67 | capv1_31_100917 | TGTCCCTCTTAGTACACGTCCTTCTACAGT |
| 68 | capv1_31_100919 | TCCCTCTTAGTACACGTCCTTCTACAGTAT |
| 69 | hpv31_cap_104028 | TGTTTAAACATGCTAGTACAACTATGCTGATGCAGTAGTTC |
| 70 | capv1_31_104028 | TTAAACATGCTAGTACAACTATGCTGATGC |
| | Mediator probes | |
| 71 | medv1_31_100961 | TACCTATTAGACCACCAGTTAGCATTGACC |
| 72 | medv1_31_100963 | CCTATTAGACCACCAGTTAGCATTGACCCT |
| 73 | medv1_31_100964 | CTATTAGACCACCAGTTAGCATTGACCCTG |
| 74 | medv1_31_100965 | TATTAGACCACCAGTTAGCATTGACCCTGT |
| 75 | medv1_31_112000 | GTTTCCTGCCTAACACACCTTGCCAACATATAATCCAGTC |
| 76 | medv1_31_112001 | TTTCCTGCCTAACACACCTTGCCAACATATAATCCAGTCC |
| 77 | medv1_31_112002 | TTCCTGCCTAACACACCTTGCCAACATATAATCCAGTCCA |
| 78 | medv1_31_104118 | TTTCCTGCCTAACACACCTTGCCAACATAT |

TABLE 4
HPV primers and probes for amplifying and typing HPV type 33

| SEQ ID NO: | | Sequence (5′-3′) |
|---|---|---|
| | PCR primers | |
| 79 | hpv33_54464_53endtrunc | TATTACATCTCGTAGACATACTGT |
| 80 | hpv33_54465_3endtrunc | GCTATTACATCTCGTAGACATACT |
| 81 | hpv33_54466_3endtrunc | CTATTACATCTCGTAGACATACTG |
| 82 | hpv33_55139_5endtrunc_rc | GTACCAATAATTTTTTAGCGTTAG |
| | Capture probes | |
| 83 | hpv33_cap_54589 | TATTGTGCCTTTAGACCACACCGTGCCAAATGAACAATATG |
| 84 | hpv33_cap_54589_extend_5 | TGCCTTTAGACCACACCGTGCCAAATGAACAAT |
| 85 | capv1_33_54588 | GTGCCTTTAGACCACACCGTGCCAAATGAA |
| 86 | capv1_33_54589 | TGCCTTTAGACCACACCGTGCCAAATGAAC |
| 87 | capv1_33_54590 | GCCTTTAGACCACACCGTGCCAAATGAACA |
| 88 | capv1_33_54591 | CCTTTAGACCACACCGTGCCAAATGAACAA |
| 89 | capv1_33_54592 | CTTTAGACCACACCGTGCCAAATGAACAAT |
| 90 | hpv33_cap_54824 | GATATACCTTCCCCTTTATTTCCCACATCTAGCCCATTTGT |
| 91 | capv1_33_54823 | TACCTTCCCCTTTATTTCCCACATCTAGCC |
| 92 | capv1_33_54824 | ACCTTCCCCTTTATTTCCCACATCTAGCCC |
| 93 | capv1_33_54833 | TTTATTTCCCACATCTAGCCCATTTGTTCC |
| 94 | capv1_33_54835 | TATTTCCCACATCTAGCCCATTTGTTCCTA |
| | Mediator probes | |
| 95 | medv1_33_54629 | ACAGCCTTTACATGATACTTCTACATCGTC |
| 96 | medv1_33_54630 | CAGCCTTTACATGATACTTCTACATCGTCT |
| 97 | medv1_33_54631 | AGCCTTTACATGATACTTCTACATCGTCTT |
| 98 | medv1_33_54876 | TTTCCTTTTGACACCATTGTTGTAGACGGT |

TABLE 5
HPV primers and probes for amplifying and typing HPV type 35

| SEQ ID NO: | | Sequence (5′-3′) |
|---|---|---|
| | PCR primers | |
| 99 | hpv35_116922_3endtrunc | TGACATCCATAAGTACACATGATA |
| 100 | hpv35_116923_3endtrunc | GACATCCATAAGTACACATGATAA |
| 101 | hpv35_117465_5endtrunc_rc | CATGTTGTAAGGGTTGTAATTCTA |
| 102 | hpv35_117509_5endtrunc_rc | ATTTAATGATGTTGAAACAGTGGT |
| | Capture probes | |
| 103 | hpv35_cap_125005 | TGTTGACCCTGCCTTTATGACTTCTCCTGCAAAACTTATTACATATGATAA |
| 104 | hpv35_cap_125005_extend_6 | GACCCTGCCTTTATGACTTCTCCTGCAAAACTTATTACATATG |
| 105 | capv1_35_125004 | GACCCTGCCTTTATGACTTCTCCTGCAAAACTTATTACAT |
| 106 | capv1_35_125005 | ACCCTGCCTTTATGACTTCTCCTGCAAAACTTATTACATA |
| 107 | capv1_35_117184 | CCCTGCCTTTATGACTTCTCCTGCAAAACT |
| 108 | capv1_35_117185 | CCTGCCTTTATGACTTCTCCTGCAAAACTT |
| 109 | capv1_35_117186 | CTGCCTTTATGACTTCTCCTGCAAAACTTA |
| 110 | capv1_35_117187 | TGCCTTTATGACTTCTCCTGCAAAACTTAT |
| 111 | capv1_35_117188 | GCCTTTATGACTTCTCCTGCAAAACTTATT |
| | Mediator probes | |
| 112 | medv1_35_125066 | CCTTAACCCTGATACAACCTTACAATTTGAGCATGAGGAT |
| 113 | medv1_35_125068 | TTAACCCTGATACAACCTTACAATTTGAGCATGAGGATAT |
| 114 | medv1_35_125070 | AACCCTGATACAACCTTACAATTTGAGCATGAGGATATTA |
| 115 | medv1_35_125073 | CCTGATACAACCTTACAATTTGAGCATGAGGATATTAGCT |

TABLE 6
HPV primers and probes for amplifying and typing HPV type 39

| SEQ ID NO: | | Sequence (5′-3′) |
|---|---|---|
| | PCR primers | |
| 116 | hpv39_205196 | CAGTAAGGTTTAGTAGGCTTGGCAAAAAGG |
| 117 | hpv39_205322_5endtrunc | TAGTTCACGCTGAGCCCTCTGATG |
| 118 | hpv39_205714_53endtrunc_rc | ATGCTGTCACTAGACCGCCACATA |
| | Capture probes | |
| 119 | hpv39_cap_213212 | TAGATACTGCATTTAATAATACAAGGGATTCGGGCACTACATATAACACAG |
| 120 | hpv39_cap_213212_extend_3 | TGCATTTAATAATACAAGGGATTCGGGCACTACATATAACACAG |
| 121 | capv1_39_205408 | ACTGCATTTAATAATACAAGGGATTCGGGC |
| 122 | capv1_39_213212 | ACTGCATTTAATAATACAAGGGATTCGGGCACTACATATA |
| 123 | capv1_39_205414 | TTTAATAATACAAGGGATTCGGGCACTACA |
| 124 | capv1_39_205415 | TTAATAATACAAGGGATTCGGGCACTACAT |
| 125 | capv1_39_205420 | AATACAAGGGATTCGGGCACTACATATAAC |
| 126 | medv1_39_205568 | GTACTACTCCACAGTTGCCATTGGTGCCTT |
| 127 | medv1_39_205569 | TACTACTCCACAGTTGCCATTGGTGCCTTC |
| 128 | medv1_39_205570 | ACTACTCCACAGTTGCCATTGGTGCCTTCT |
| 129 | medv1_39_205571 | CTACTCCACAGTTGCCATTGGTGCCTTCTG |
| 130 | medv1_39_205572 | TACTCCACAGTTGCCATTGGTGCCTTCTGG |
| | Mediator probes | |
| 131 | medv1_39_205568 | GTACTACTCCACAGTTGCCATTGGTGCCTT |
| 132 | medv1_39_205569 | TACTACTCCACAGTTGCCATTGGTGCCTTC |
| 133 | medv1_39_205570 | ACTACTCCACAGTTGCCATTGGTGCCTTCT |
| 134 | medv1_39_205571 | CTACTCCACAGTTGCCATTGGTGCCTTCTG |
| 135 | medv1_39_205572 | TACTCCACAGTTGCCATTGGTGCCTTCTGG |

TABLE 7
HPV primers and probes for amplifying and typing HPV type 45

| SEQ ID NO: | | Sequence (5′-3′) |
|---|---|---|
| | PCR primers | |
| 136 | hpv45_126840_3endtrunc | TAGGGCTAATCAACAGGTCCGTGT |
| 137 | hpv45_126840_5endtrunc | TAATCAACAGGTCCGTGTGTCCAC |
| 138 | hpv45_127941_3endtrunc | CTGTTATTACGCAGGATGTTAGGG |
| 139 | hpv45_127941_5endtrunc | TTACGCAGGATGTTAGGGATAATG |
| 140 | hpv45_127942_5endtrunc | TACGCAGGATGTTAGGGATAATGT |
| 141 | hpv45_127302_rc | AATGGTACTGTAACATTACTGTAAGAGGAT |
| 142 | hpv45_128604_3endtrunc_rc | ACTGCTTAAACTTAGTAGGGTCAT |
| 143 | hpv45_128604_rc | TACTATACTGCTTAAACTTAGTAGGGTCAT |
| | Capture probes | |
| 144 | hpv45_cap_126880 | AGTTTTTAACACATCCCTCATCGTTGGTTACATTTGATAAT |
| 145 | capv1_45_126880 | TTAACACATCCCTCATCGTTGGTTACATTT |
| 146 | capv1_45_126882 | AACACATCCCTCATCGTTGGTTACATTTGA |
| 147 | capv1_45_126883 | ACACATCCCTCATCGTTGGTTACATTTGAT |
| 148 | capv1_45_126884 | CACATCCCTCATCGTTGGTTACATTTGATA |
| 149 | hpv45_cap_127211 | TGCAGACTTCCCACCTCCTGCGTCCACTACACCTAGCACTA |
| 150 | hpv45_cap_127211_extend_2 | TGCAGACTTCCCACCTCCTGCGTCCACTACACCTAG |
| 151 | capv1_45_127211 | ACTTCCCACCTCCTGCGTCCACTACACCTA |
| 152 | hpv45_cap_128173 | TTGCAGGATACAAAGTGCGAGGTTCCATTAGACATTTGTCA |
| 153 | hpv45_cap_128173_extend_2 | TTGCAGGATACAAAGTGCGAGGTTCCATTAGACATT |
| 154 | capv1_45_128173 | GGATACAAAGTGCGAGGTTCCATTAGACAT |
| | Mediator probes | |
| 155 | medv1_45_126936 | CACCACACTATCCTTTGAGCCTACCAGTAA |
| 156 | medv1_45_126937 | ACCACACTATCCTTTGAGCCTACCAGTAAT |
| 157 | medv1_45_126938 | CCACACTATCCTTTGAGCCTACCAGTAATG |
| 158 | medv1_45_127265 | ATCCAAAGTATTCCTTGACCATGCCTTCTA |
| 159 | medv1_45_127269 | AAAGTATTCCTTGACCATGCCTTCTACTGC |
| 160 | medv1_45_127271 | AGTATTCCTTGACCATGCCTTCTACTGCTG |
| 161 | medv1_45_128326 | TGTTATGGGTGACACAGTACCTACGGACCT |

TABLE 8
HPV primers and probes for amplifying and typing HPV type 51

| SEQ ID NO: | | Sequence (5′-3′) |
|---|---|---|
| | PCR primers | |
| 162 | hpv51_215984_3endtrunc | GAGAGTATAGACGTTATAGCAGGT |
| 163 | hpv51_220678_5endtrunc | CAGTACGCTTTAGTAGGTTAGGTC |
| 164 | hpv51_216387_3endtrunc_rc | ACGGAGCTTCAATTCTGTAACACG |
| 165 | hpv51_216387_53endtrunc_rc | AACACGGAGCTTCAATTCTGTAAC |
| 166 | hpv51_221289_3endtrunc_rc | AGTCTGGAACTGCCTGCATAGTAA |
| 167 | hpv51_221289_53endtrunc_rc | ATTAGTCTGGAACTGCCTGCATAG |
| | Capture probes | |
| 168 | hpv51_cap_216256 | AAAGATGTAGTATTGCATTTAACACCACAGACTGAAATTGA |
| 169 | hpv51_cap_216256_extend_1 | AAAGATGTAGTATTGCATTTAACACCACAGACTGAAATTGA |
| 170 | capv1_51_216256 | TGTAGTATTGCATTTAACACCACAGACTGA |
| 171 | capv1_51_216257 | GTAGTATTGCATTTAACACCACAGACTGAA |
| 172 | hpv51_cap_220901 | CACACCACACCTATGTCACACTCCTCTTTGTCTAGGCAGTT |
| 173 | hpv51_cap_220901_extend_1 | CACACCACACCTATGTCACACTCCTCTTTGTCTAGGCAGTT |
| 174 | capv1_51_220899 | ACCACACCTATGTCACACTCCTCTTTGTCT |
| 175 | capv1_51_220901 | CACACCTATGTCACACTCCTCTTTGTCTAG |
| | Mediator probes | |
| 176 | medv1_51_216346 | TATGCGTGACCAGCTACCAGAAAGACGGGC |
| 177 | medv1_51_221069 | TCCCCACACTTCCATTGACACCAAGCATTC |
| 178 | medv1_51_221072 | CCACACTTCCATTGACACCAAGCATTCTAT |
| 179 | medv1_51_228853 | ACACTTCCATTGACACCAAGCATTCTATTGTTATACTAGG |
| 180 | medv1_51_221075 | CACTTCCATTGACACCAAGCATTCTATTGT |
| 181 | medv1_51_221080 | CCATTGACACCAAGCATTCTATTGTTATAC |

TABLE 9
HPV primers and probes for amplifying and typing HPV type 56

| SEQ ID NO: | | Sequence (5′-3′) |
|---|---|---|
| | PCR primers | |
| 182 | hpv56_142266_3endtrunc | ATCCGTTATTTATTGATCCCCCTG |
| 183 | hpv56_142266_53endtrunc | CGTTATTTATTGATCCCCCTGTTA |
| 184 | hpv56_142689_53endtrunc_rc | TACGTGTTTGTATAGTAGCCTTTC |
| 185 | hpv56_142690_5endtrunc_rc | CCTCTACGTGTTTGTATAGTAGCC |
| | Capture probes | |
| 186 | hpv56_cap_142497 | TAAGGTAACTGACCCTGCATTTCTTGATAGACCTGCAACATTAGTATCTGCTGAT |
| 187 | hpv56_cap_142497_extend_4 | GGTAACTGACCCTGCATTTCTTGATAGACCTGCAAC |
| 188 | capv1_56_142495 | GGTAACTGACCCTGCATTTCTTGATAGACC |
| 189 | capv1_56_142496 | GTAACTGACCCTGCATTTCTTGATAGACCT |
| 190 | capv1_56_142497 | TAACTGACCCTGCATTTCTTGATAGACCTG |
| 191 | capv1_56_142498 | AACTGACCCTGCATTTCTTGATAGACCTGC |
| 192 | capv1_56_142508 | GCATTTCTTGATAGACCTGCAACATTAGTA |
| 193 | capv1_56_142509 | CATTTCTTGATAGACCTGCAACATTAGTAT |
| 194 | capv1_56_142511 | TTTCTTGATAGACCTGCAACATTAGTATCT |
| 195 | capv1_56_142512 | TTCTTGATAGACCTGCAACATTAGTATCTG |
| | Mediator probes | |
| 196 | medv1_56_142563 | GTACTGACACATCTTTAGCTTTTTCTCCGT |
| 197 | medv1_56_142566 | CTGACACATCTTTAGCTTTTTCTCCGTCGG |
| 198 | medv1_56_142567 | TGACACATCTTTAGCTTTTTCTCCGTCGGG |
| 199 | medv1_56_142568 | GACACATCTTTAGCTTTTTCTCCGTCGGGT |

TABLE 10
HPV primers and probes for amplifying and typing HPV type 58

| SEQ ID NO: | | Sequence (5′-3′) |
|---|---|---|
| | PCR primers | |
| 200 | hpv58_65683_5endtrunc | GTGTAACCTGTAACAACGCCATGA |
| 201 | hpv58_70331_3endtrunc | TAGGGGCTAAAGTACATTACTACC |
| 202 | hpv58_70332_3endtrunc | AGGGGCTAAAGTACATTACTACCA |
| 203 | hpv58_66521_5endtrunc_rc | CTGTCTACATCCGTTTCACTGCTT |
| 204 | hpv58_66523_5endtrunc_rc | AACTGTCTACATCCGTTTCACTGC |
| 205 | hpv58_78723_53endtrunc_rc | ACTTTTTTATTGTTATTGGGACTT |
| 206 | hpv58_78723_5endtrunc_rc | ATTGTTATTGGGACTTTTGATGGA |
| | Capture probes | |
| 207 | hpv58_cap_66078 | AGGTAGAAGCGGTAATAGAACGAAGAACAGGAGATAATATT |
| 208 | capv1_58_66078 | GAAGCGGTAATAGAACGAAGAACAGGAGAT |
| 209 | capv1_58_74314 | TCAGATGTAAGCAGTGAAACGGATGTAGACAGTTGTAATA |
| 210 | capv1_58_74315 | CAGATGTAAGCAGTGAAACGGATGTAGACAGTTGTAATAC |
| 211 | capv1_58_66521 | AGATGTAAGCAGTGAAACGGATGTAGACAG |
| 212 | capv1_58_74316 | AGATGTAAGCAGTGAAACGGATGTAGACAGTTGTAATACT |
| 213 | capv1_58_66522 | GATGTAAGCAGTGAAACGGATGTAGACAGT |
| 214 | capv1_58_74317 | GATGTAAGCAGTGAAACGGATGTAGACAGTTGTAATACTG |
| 215 | capv1_58_66523 | ATGTAAGCAGTGAAACGGATGTAGACAGTT |
| 216 | capv1_58_66524 | TGTAAGCAGTGAAACGGATGTAGACAGTTG |
| 217 | capv1_58_66525 | GTAAGCAGTGAAACGGATGTAGACAGTTGT |
| 218 | capv1_58_66526 | TAAGCAGTGAAACGGATGTAGACAGTTGTA |
| 219 | capv1_58_66527 | AAGCAGTGAAACGGATGTAGACAGTTGTAA |
| 220 | capv1_58_66528 | AGCAGTGAAACGGATGTAGACAGTTGTAAT |
| 221 | capv1_58_70473 | TTATGCTGACGATGCTGATACTATACATGA |
| 222 | hpv58_cap_70587 | TTTGACACTCCTCTTGTGTCATTGGAACCTGGTCCAGACAT |
| 223 | hpv58_cap_70587_extend_3 | CACTCCTCTTGTGTCATTGGAACCTGGTCCAGACAT |
| 224 | capv1_58_70586 | ACACTCCTCTTGTGTCATTGGAACCTGGTC |
| 225 | capv1_58_70587 | CACTCCTCTTGTGTCATTGGAACCTGGTCC |
| | Mediator probes | |
| 226 | medv1_58_65889 | ACTTGTGGCACCACGGTTCGTTTGTGTATC |
| 227 | medv1_58_65890 | CTTGTGGCACCACGGTTCGTTTGTGTATCA |
| 228 | medv1_58_66290 | ATGCTCAGAAAGTGCTGTAGAGGACTGTGT |
| 229 | medv1_58_70648 | AGTCCATTTATTCCTATATCTCCACTAACT |
| 230 | medv1_58_70650 | TCCATTTATTCCTATATCTCCACTAACTCC |

TABLE 11
HPV primers and probes for amplifying and typing HPV type 59

| SEQ ID NO: | | Sequence (5′-3′) |
|---|---|---|
| | PCR primers | |
| 231 | hpv59_158129_53endtrunc | CTGACTTTTTAACACGTCCATCCA |
| 232 | hpv59_158274_53endtrunc | AACATCCAGACGCAGCACTGTAAG |
| 233 | hpv59_160491_5endtrunc | TGTCCCTTTATTGTTTCTTTGTCC |
| 234 | hpv59_160492_5endtrunc | GTCCCTTTATTGTTTCTTTGTCCT |
| 235 | hpv59_158666_rc | TGGTAAAGGGTGTAGTAGAATAAGTGGGTT |
| 236 | hpv59_158681_3endtrunc_rc | ACTGTATGGTGGTAAAGGGTGTAG |
| 237 | hpv59_160920_3endtrunc_rc | TTCTTGGATTGCACAGTAGTTTTG |
| 238 | hpv59_168789_5endtrunc_rc | CATTCTTGGATTGCACAGTAGTTT |
| | Capture probes | |
| 239 | hpv59_cap_158382 | CATGATATAAGCCCTATACCACATGCTGAAGATATTGAATT |
| 240 | hpv59_cap_158382_extend_3 | TATAAGCCCTATACCACATGCTGAAGATATTGAATT |
| 241 | capv1_59_158382 | TATAAGCCCTATACCACATGCTGAAGATAT |
| 242 | capv1_59_158383 | ATAAGCCCTATACCACATGCTGAAGATATT |
| 243 | capv1_59_158384 | TAAGCCCTATACCACATGCTGAAGATATTG |
| 244 | capv1_59_158386 | AGCCCTATACCACATGCTGAAGATATTGAA |
| 245 | capv1_59_158387 | GCCCTATACCACATGCTGAAGATATTGAAT |
| 246 | hpv59_cap_160734 | AATCCCCATCTTGTTTCCTCCTACACGCCTAGACTACTAAC |
| 247 | hpv59_cap_160734_extend_2 | AATCCCCATCTTGTTTCCTCCTACACGCCTAGACTA |
| 248 | capv1_59_160734 | CCATCTTGTTTCCTCCTACACGCCTAGACT |
| | Mediator probes | |
| 249 | medv1_59_158467 | TATGCAGATATTACAGATGAAGCACCTACT |
| 250 | medv1_59_158468 | ATGCAGATATTACAGATGAAGCACCTACTA |
| 251 | medv1_59_158469 | TGCAGATATTACAGATGAAGCACCTACTAG |
| 252 | medv1_59_160767 | AACACAACTTACAAACGCCAAATAGTTAGT |
| 253 | medv1_59_160769 | CACAACTTACAAACGCCAAATAGTTAGTCA |
| 254 | medv1_59_160770 | ACAACTTACAAACGCCAAATAGTTAGTCAT |

TABLE 12
HPV primers and probes for amplifying and typing HPV type 66

| SEQ ID NO: | | Sequence (5′-3′) |
|---|---|---|
| | PCR primers | |
| 255 | hpv66_176255_53endtrunc | GTAGTATCCTTGGGCAGTGTGTGT |
| 256 | hpv66_176255_5endtrunc | GTATCCTTGGGCAGTGTGTGTCAG |
| 257 | hpv66_176522_5endtrunc_rc | TAGTTGGCACAGAAATACAGGTGA |
| 258 | hpv66_176522_rc | TAGTTGGCACAGAAATACAGGTGAGTAATA |
| | Capture probes | |
| 259 | hpv66_cap_176349 | AGTAACACACCAAACTCCATTTTAGTGCTGTACGCCATTTT |
| 260 | hpv66_cap_176349_extend_4 | AACACACCAAACTCCATTTTAGTGCTGTACGCC |
| 261 | capv1_66_176347 | AACACACCAAACTCCATTTTAGTGCTGTAC |
| 262 | capv1_66_176348 | ACACACCAAACTCCATTTTAGTGCTGTACG |
| 263 | capv1_66_176349 | CACACCAAACTCCATTTTAGTGCTGTACGC |
| 264 | capv1_66_176350 | ACACCAAACTCCATTTTAGTGCTGTACGCC |
| | Mediator probes | |
| 265 | medv1_66_176398 | AATTCGGTTGCCTAGCCTTTTGTCCTTATT |
| 266 | medv1_66_176399 | ATTCGGTTGCCTAGCCTTTTGTCCTTATTT |
| 267 | medv1_66_176400 | TTCGGTTGCCTAGCCTTTTGTCCTTATTTA |

TABLE 13
HPV primers and probes for amplifying and typing HPV type 68

| SEQ ID NO: | | Sequence (5′-3′) |
|---|---|---|
| | PCR primers | |
| 268 | hpv68_185502_5endtrunc | ACAGCACAGGTACTTTTGAATATG |
| 269 | hpv68_185503_53endtrunc | AGACAGCACAGGTACTTTTGAATA |
| 270 | hpv68_189645_53endtrunc | ATGCACCTGATACTGACAATACTA |
| 271 | hpv68_191001_5endtrunc | ACATTGTCCACTACTACAGACTCT |
| 272 | hpv68_185878_5endtrunc_rc | ACATTGCAGCCTTTTTATTGTTAC |
| 273 | hpv68_190079_5endtrunc_rc | AATAACCTAGATGTACCAGCATAG |
| 274 | hpv68_190082_5endtrunc_rc | GTTAATAACCTAGATGTACCAGCA |
| 275 | hpv68_191484_rc | CAAGACATATAACAATTATTTTGACACACG |
| | Capture probes | |
| 276 | capv1_68_184546 | GCAGGACATTGGACACTACATTGCATGACG |
| 277 | capv1_68_184546 | GCAGGACATTGGACACTACATTGCATGACG |
| 278 | capv1_68_184549 | GGACATTGGACACTACATTGCATGACGTTA |
| 279 | capv1_68_184549 | GGACATTGGACACTACATTGCATGACGTTA |
| 280 | hpv68_cap_185604 | ATAGAAAGCAGTCCTTTAGCAAAGTCGCCATTACAGGAATTATCACTA |
| 281 | hpv68_cap_185604_extend_6 | AAGCAGTCCTTTAGCAAAGTCGCCATTACAGGAATTA |
| 282 | capv1_68_185597 | AAGCAGTCCTTTAGCAAAGTCGCCATTACA |
| 283 | capv1_68_185604 | CCTTTAGCAAAGTCGCCATTACAGGAATTA |
| 284 | capv1_68_185604 | CCTTTAGCAAAGTCGCCATTACAGGAATTA |
| 285 | capv1_68_185605 | CTTTAGCAAAGTCGCCATTACAGGAATTAT |
| 286 | capv1_68_185605 | CTTTAGCAAAGTCGCCATTACAGGAATTAT |
| 287 | capv1_68_189702 | CATTTACTACTCGTTCCCACATATCAGTTC |
| 288 | capv1_68_189703 | ATTTACTACTCGTTCCCACATATCAGTTCC |
| 289 | capv1_68_189726 | CAGTTCCTTCATTGGCTTCTGCTGCATCCA |
| 290 | capv1_68_189727 | AGTTCCTTCATTGGCTTCTGCTGCATCCAC |
| 291 | capv1_68_189729 | TTCCTTCATTGGCTTCTGCTGCATCCACTA |
| 292 | capv1_68_189730 | TCCTTCATTGGCTTCTGCTGCATCCACTAC |
| 293 | capv1_68_189732 | CTTCATTGGCTTCTGCTGCATCCACTACAT |
| 294 | capv1_68_189733 | TTCATTGGCTTCTGCTGCATCCACTACATA |
| 295 | capv1_68_189738 | TGGCTTCTGCTGCATCCACTACATATACTA |
| 296 | hpv68_cap_191232 | TAGATACATACCGCTACCTACAATCAGCAGCAATTACATGT |
| 297 | hpv68_cap_191232_extend_1 | TAGATACATACCGCTACCTACAATCAGCAGCAATTACATGT |
| 298 | capv1_68_191232 | ACATACCGCTACCTACAATCAGCAGCAATT |
| 299 | capv1_68_191269 | AAAAGGACGCCCCTGCACCTGTTAAAAAAG |
| | Mediator probes | |
| 300 | medv1_68_184624 | TATATGAATTTGCCTTTAGTGACCTATGTG |
| 301 | medv1_68_184624 | TATATGAATTTGCCTTTAGTGACCTATGTG |
| 302 | medv1_68_184627 | ATGAATTTGCCTTTAGTGACCTATGTGTAG |
| 303 | medv1_68_184627 | ATGAATTTGCCTTTAGTGACCTATGTGTAG |
| 304 | medv1_68_185707 | AAGTGGAAACTAACTCGGAGGTAACTGTAG |
| 305 | medv1_68_185841 | GATCCTAAATCACCTACTACCCAACTTAAA |
| 306 | medv1_68_185841 | GATCCTAAATCACCTACTACCCAACTTAAA |
| 307 | medv1_68_185844 | CCTAAATCACCTACTACCCAACTTAAAGTA |
| 308 | medv1_68_185844 | CCTAAATCACCTACTACCCAACTTAAAGTA |
| 309 | medv1_68_189732 | CTTCATTGGCTTCTGCTGCATCCACTACAT |
| 310 | medv1_68_189733 | TTCATTGGCTTCTGCTGCATCCACTACATA |
| 311 | medv1_68_189845 | CCACAGTTGCCTTTAACACCCTCTACTCCA |
| 312 | medv1_68_189847 | ACAGTTGCCTTTAACACCCTCTACTCCAAT |
| 313 | medv1_68_189848 | CAGTTGCCTTTAACACCCTCTACTCCAATT |

### Results

The following analytical and clinical sample data represent results demonstrating the utility of co-amplifying a control DNA of known input copy number into a multiplex PCR for specific HPV subtype amplification and detection. Table 14 contains the PCR primers, capture probes, and mediator probes used in these experiments.

### Analytical Data

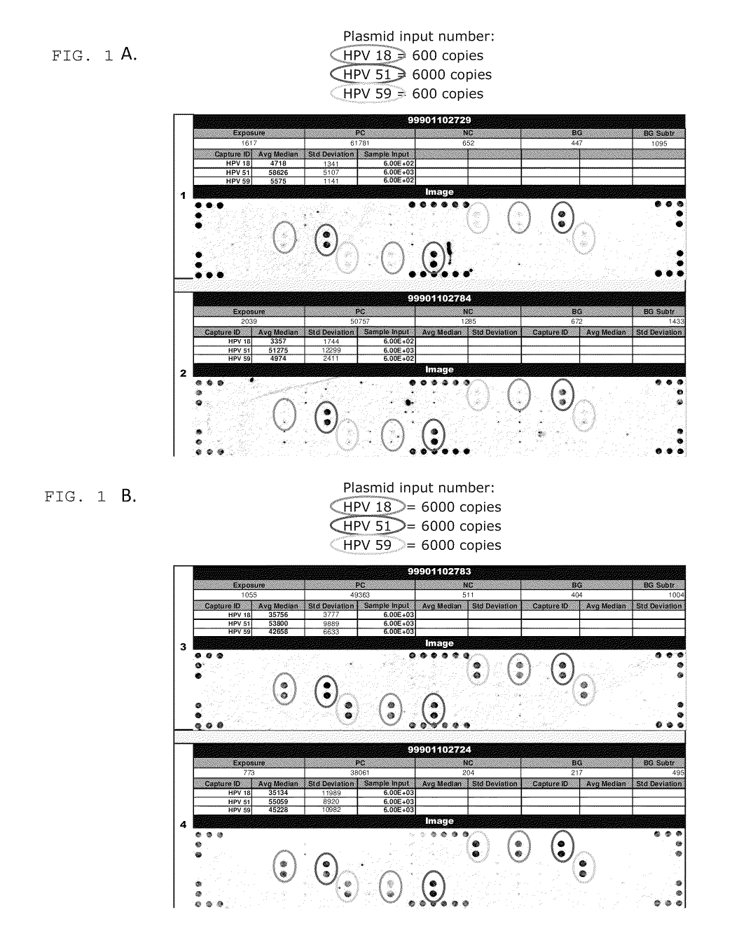

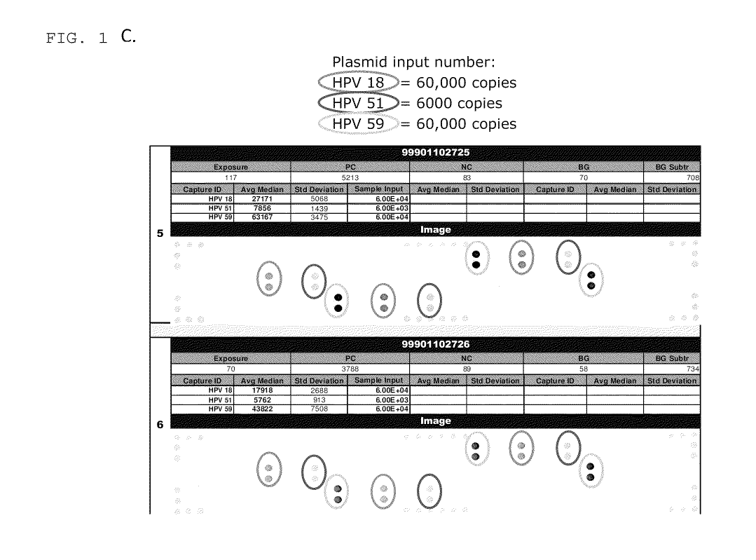

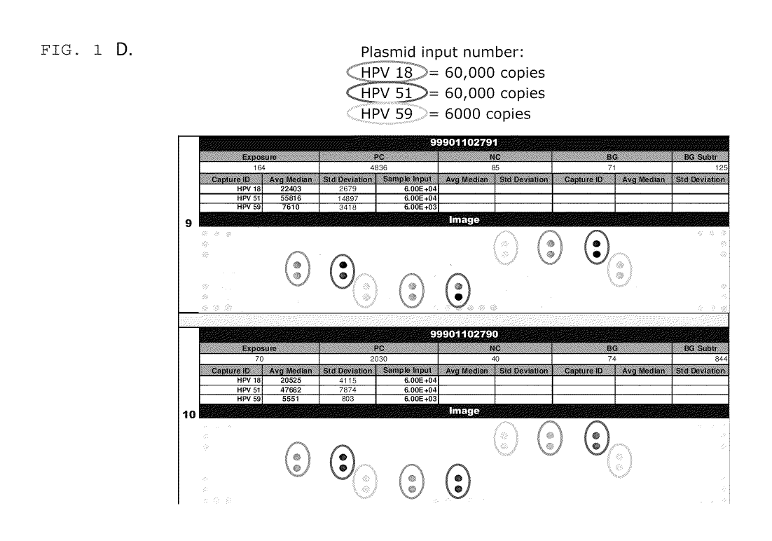

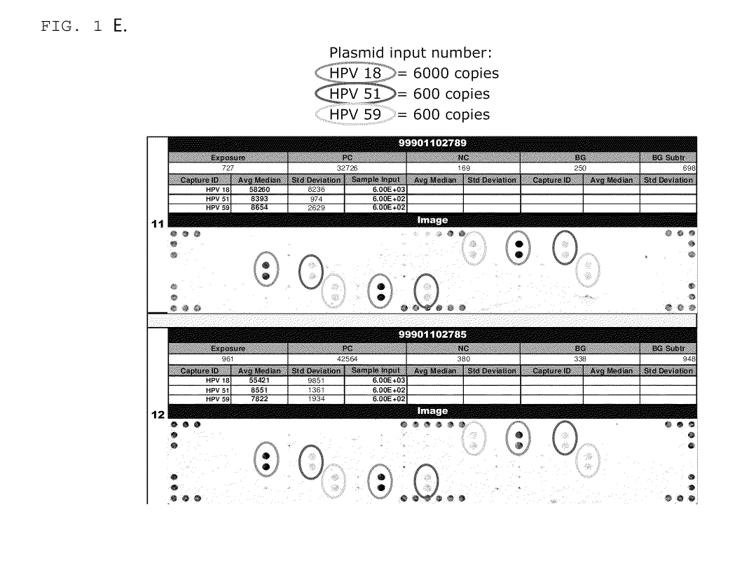

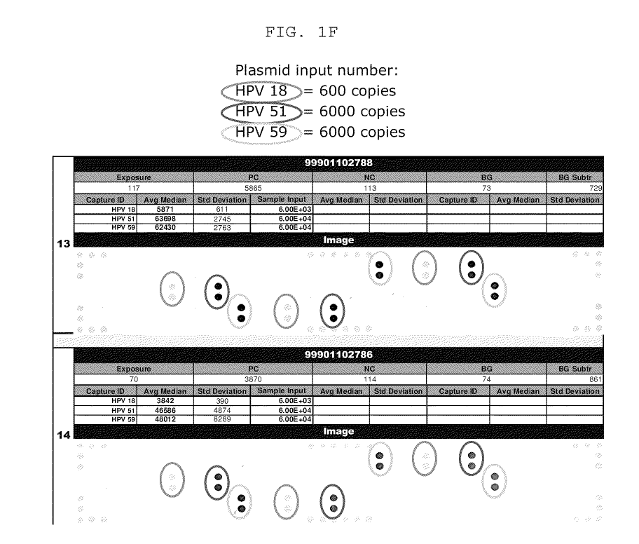

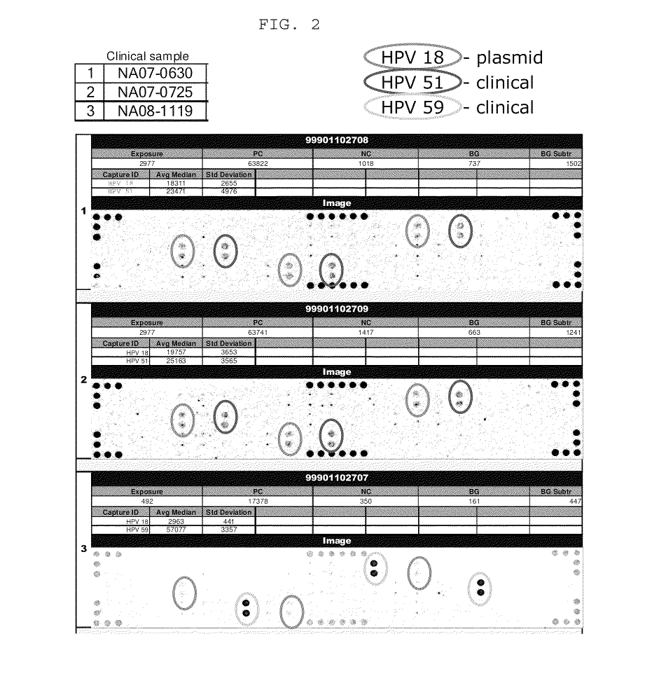

The sample mixtures in these experiments contain known input copy numbers of specific HPV subtype plasmids, specifically, for subtypes 18, 51, and 59. These samples were amplified in a multiplex PCR mixture (Table 15) for a specific number of cycles that stopped the reactions during the exponential amplification phase of the PCR before reaching the linear or plateau phases (Table 16). Then an aliquot of the multiplex PCR was diluted 1:10,000 and applied to a Verigene gold nanoparticle assay. The assay cartridge slide contained capture probes (Table 14), which targeted regions of the amplified DNA. The data in FIG. 1 demonstrate the relative qualitative sensitivity of the gold nanoparticle assay based on different plasmid input copy number.

TABLE 14
Nucleotide sequences for PCR primers, capture probes, and mediator probes.

| | HPV | | PCR primers | | | |
|---|---|---|---|---|---|---|
| | subtype | Mix | Name | Sequence (5′-3′) | Tm | Amp size |
| A. | HPV 18 | 7 | hpv18_38408_3endtrunc | TATCACACCTTCGTCTACCTCTGT | 62.88 | 610 |
| | | | hpv18_38989_5endtrunc_rc | CATTGTCCTCCGTGGCAGATACTA | 64.3 | |
| | HPV 51 | 29 | hpv51_215984_3endtrunc | GAGAGTATAGACGTTATAGCAGGT | 59.22 | 432 |
| | | | hpv51_216387_3endtrunc_rc | ACGGAGCTTCAATTCTGTAACACG | 63.86 | |
| | HPV 59 | 42 | hpv59_158274_53endtrunc | AACATCCAGACGCAGCACTGTAAG | 65.67 | 421 |
| | | | hpv59_158666_rc | TGGTAAAGGGTGTAGTAGAATAAGTGGGTT | 65.75 | |

| | HPV | Capture probes (/3AmM/) | |
|---|---|---|---|
| | subtype | Name | Sequence (5′-3′) |
| B. | HPV 18 | CAPvE_18_E38693 | TAACCCTGAGTTTCTTACACGTCCATCCTC |
| | HPV 51 | CAPvE_51_E216256 | TGTAGTATTGCATTTAACACCACAGACTGA |
| | HPV 59 | CAPvE_59_E158382 | TATAAGCCCTATACCACATGCTGAAGATAT |

| | HPV | Mediator probes | |
|---|---|---|---|
| | subtype | Name | Sequence (5′-3′) |
| C. | HPV 18 | MEDv1_18_38772 | ACATTTGATCCTCGTAGTGATGTTCCTGATaaaaaaaaaaaaaaaaaaaaaaaaaaaaaaaaaaa |
| | HPV 51 | MEDv1_51_216346 | TATGCGTGACCAGCTACCAGAAAGACGGGCaaaaaaaaaaaaaaaaaaaaaaaaaaaaaaaaaaa |
| | HPV 59 | MEDv1_59_158467 | TATGCAGATATTACAGATGAAGCACCTACTaaaaaaaaaaaaaaaaaaaaaaaaaaaaaaaaaaa |

TABLE 15
PCR thermocycling parameters for
analytical samples.

| Reagents | Final Conc. |
|---|---|
| Primer Mix 7 | 0.80 μM |
| Primer Mix29 | 0.80 μM |
| Primer Mix 42 | 0.80 μM |
| HPV plasmid mix | variable |
| | type 18, 51, and 59 |
| dNTPs | 200 μM ea. |
| 10X PCR Buffer | 1X |
| MgCl₂ | 5 mM |
| Uracil N Glycosylase | I unit |
| FastStart Taq DNA Pol | 2 units |

TABLE 16
PCR thermocycling parameters
for analytical samples.

| Cycles | | Temp. (° C.) | Time (min.) |
|---|---|---|---|
| 1X | | 95 | 05:00 |
| | | 94 | 00:30 |
| 27X | {open oversize brace} | 56 | 00:30 |
| | | 72 | 01:00 |

### Clinical Data

Based on the success of the analytical data with known input copy number of plasmids, this method was applied to clinical samples. Clinical samples were selected that were previously determined to be either HPV subtype 51 or 59 and those samples were co-amplified with a low plasmid copy number for HPV subtype 18. In this method, the plasmid DNA for HPV subtype 18 could serve as the normalizing control for a known input copy number of DNA. In these experiments, the input copy number is 250 copies. If extrapolated, this would be equivalent to 12,500 copies of HPV in a clinical sample extraction. This input copy number could be adjusted to normalize the intensity data and therefore serve as a determined clinical relevant cutoff point in a Verigene gold nanoparticle assay.

For the clinical samples, the cycling parameters were adjusted to accommodate the background human genomic DNA (Table 17), but still retain the same overall PCR cycle number as the previous analytical data so that the amplification is stopped in the exponential phase. The resulting data is shown in FIG. 2.

TABLE 17
PCR thermocycling parameters
for clinical samples.

| Cycles | | Temp. (° C.) | Time (min.) |
|---|---|---|---|
| 1X | | 95 | 05:00 |
| | | 94 | 00:30 |
| 15X | {open oversize brace} | 58 | 01:30 |
| | | 72 | 01:00 |
| | | 94 | 00:30 |
| 12X | {open oversize brace} | 58 | 00:30 |
| | | 72 | 01:00 |

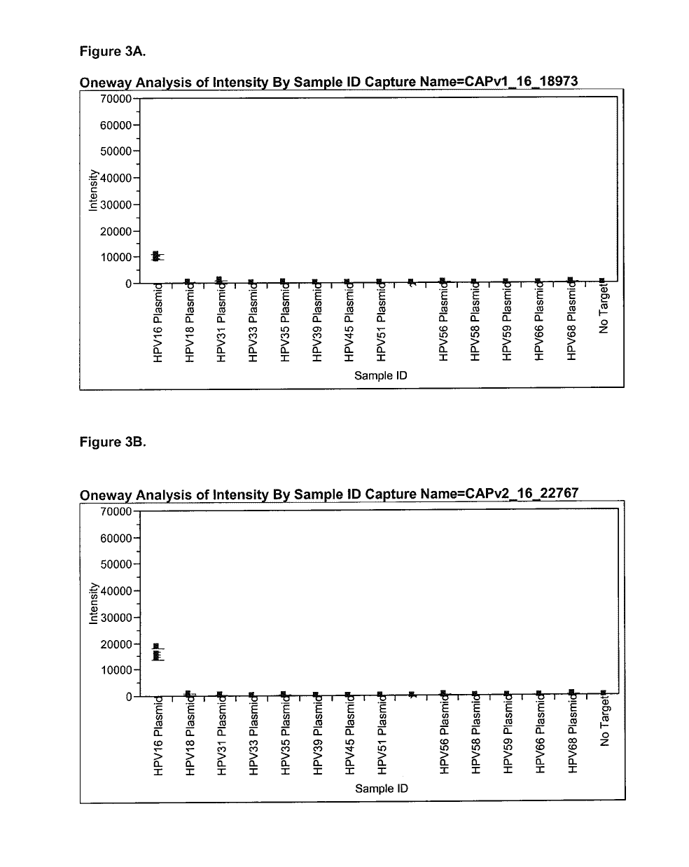

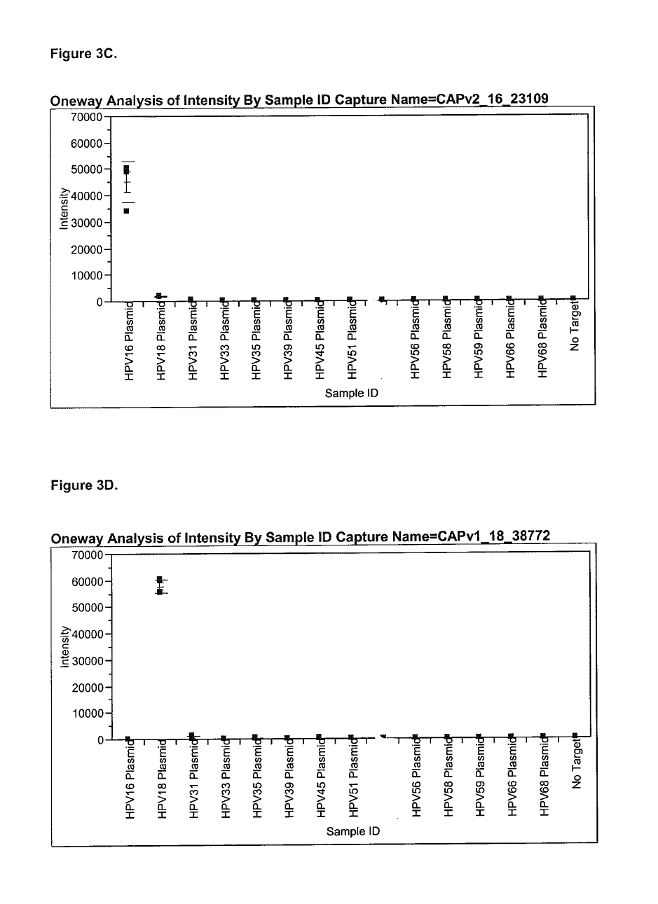

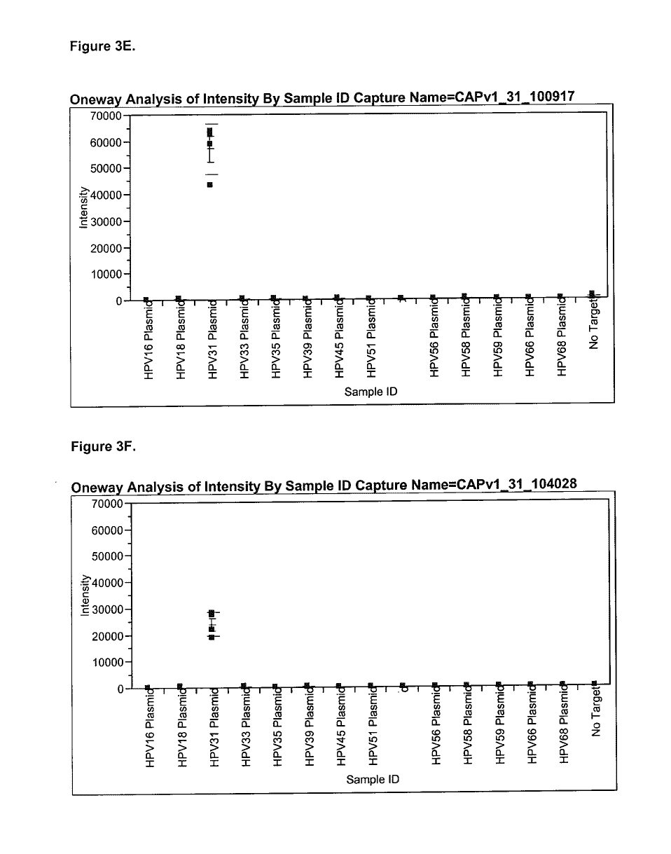

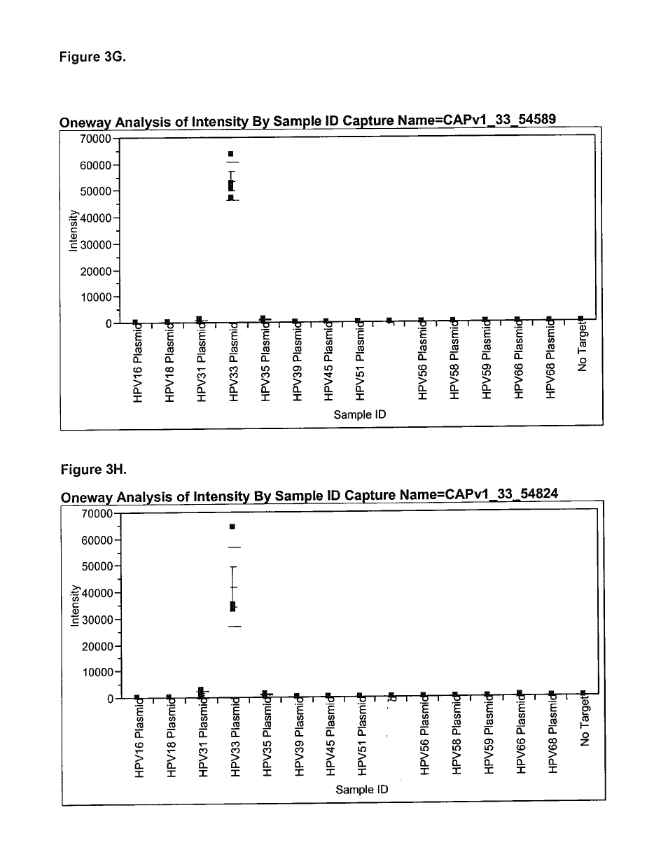

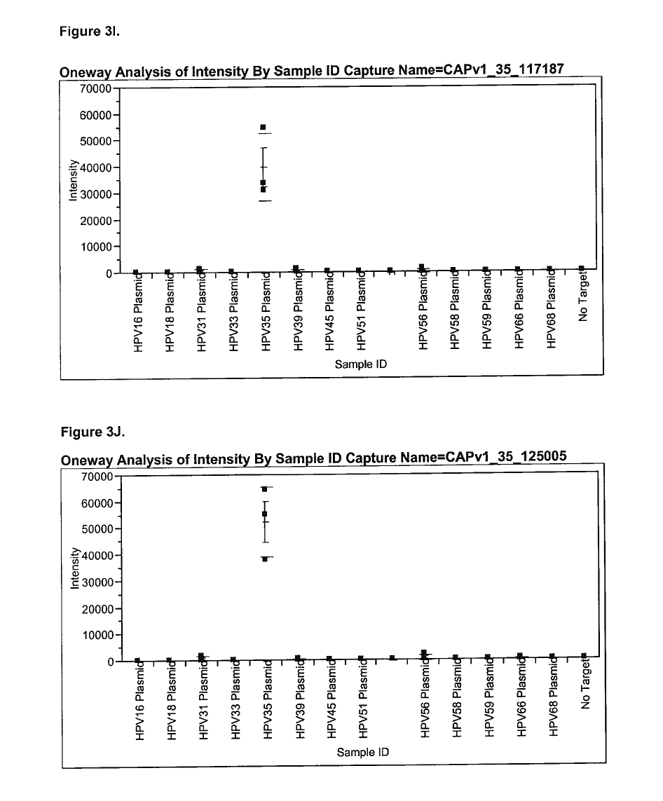

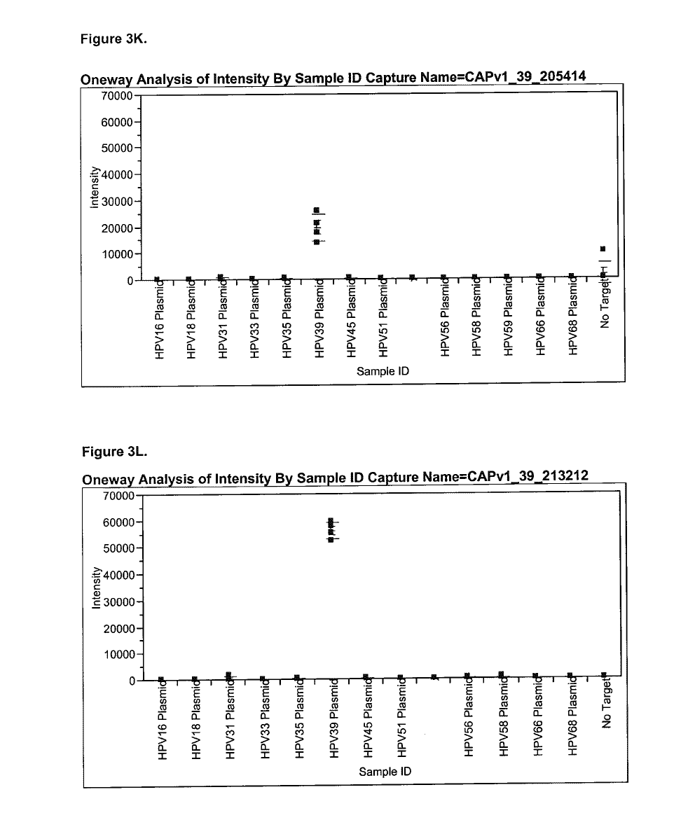

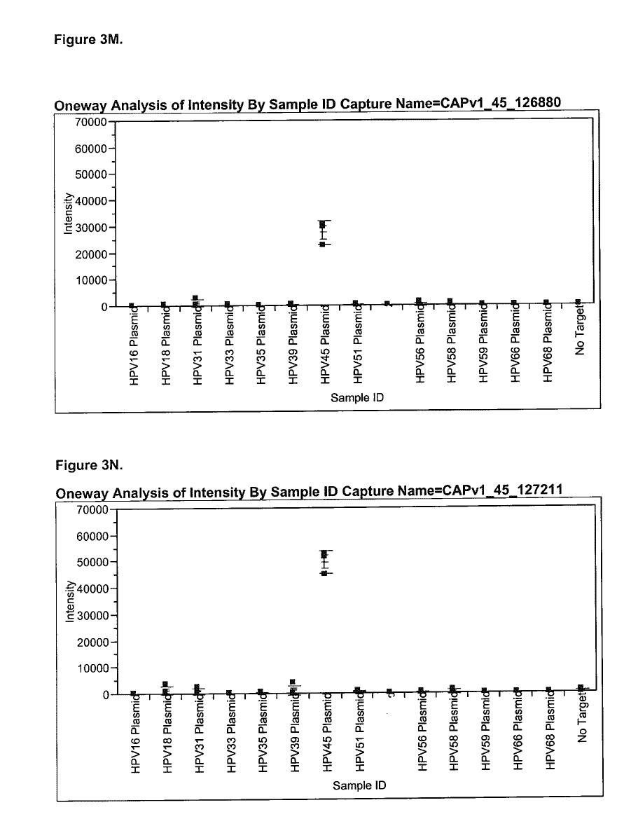

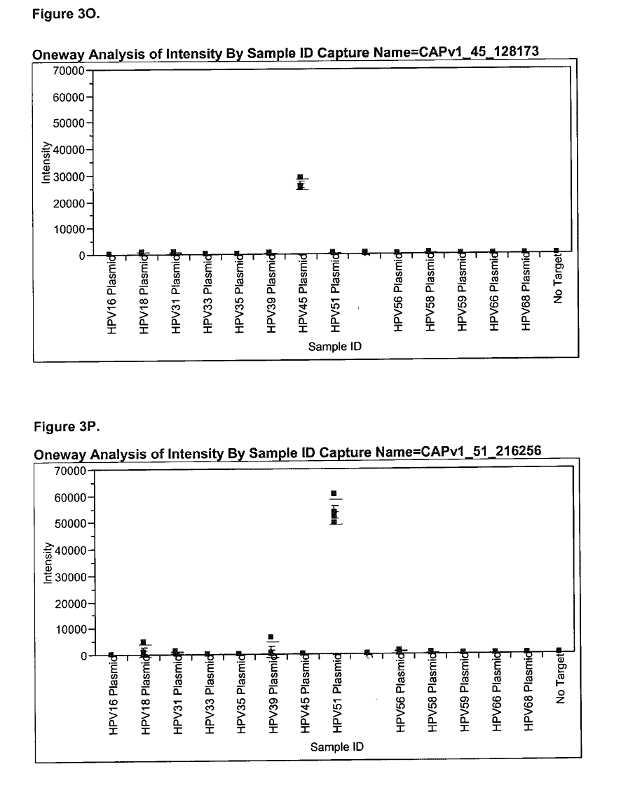

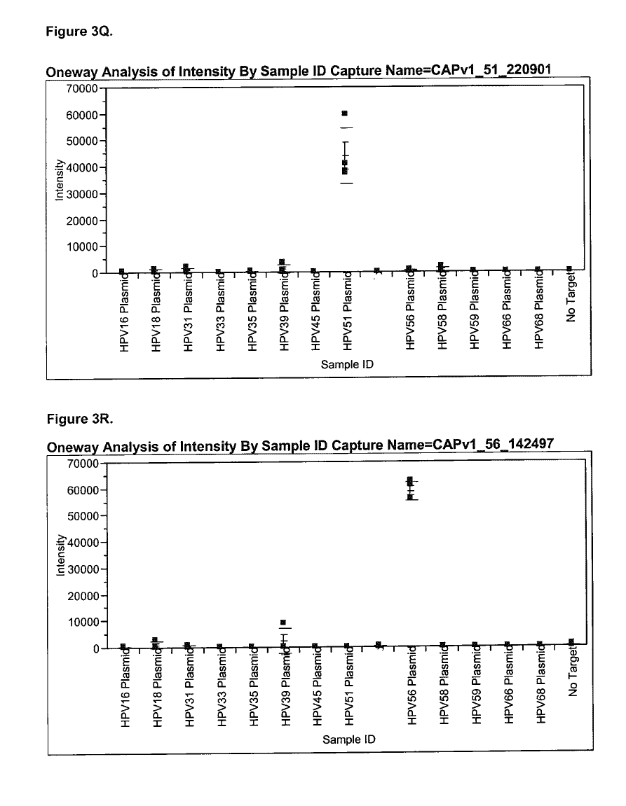

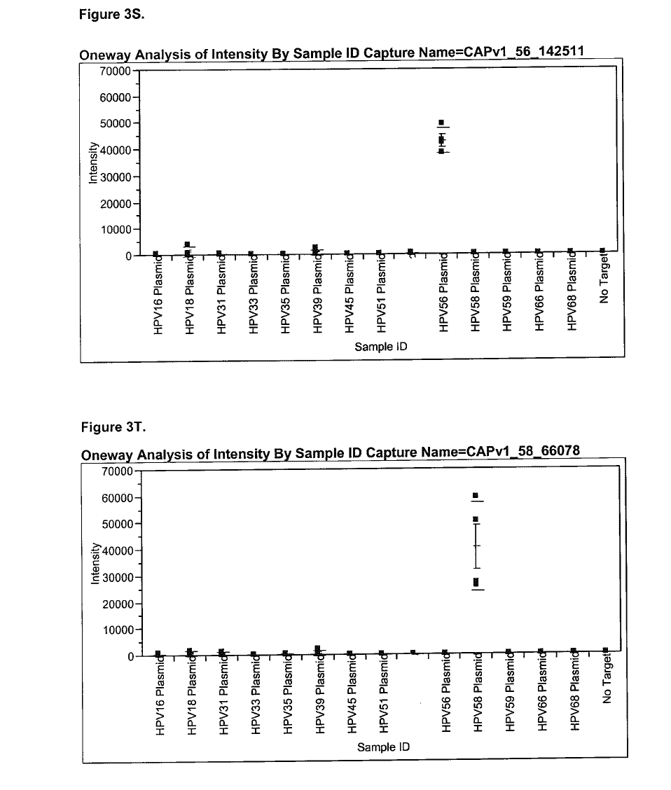

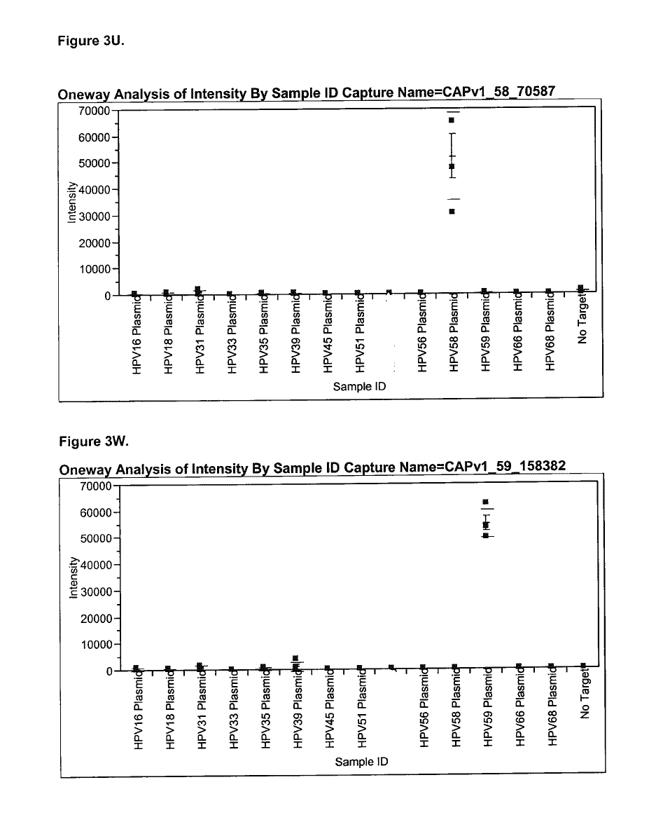

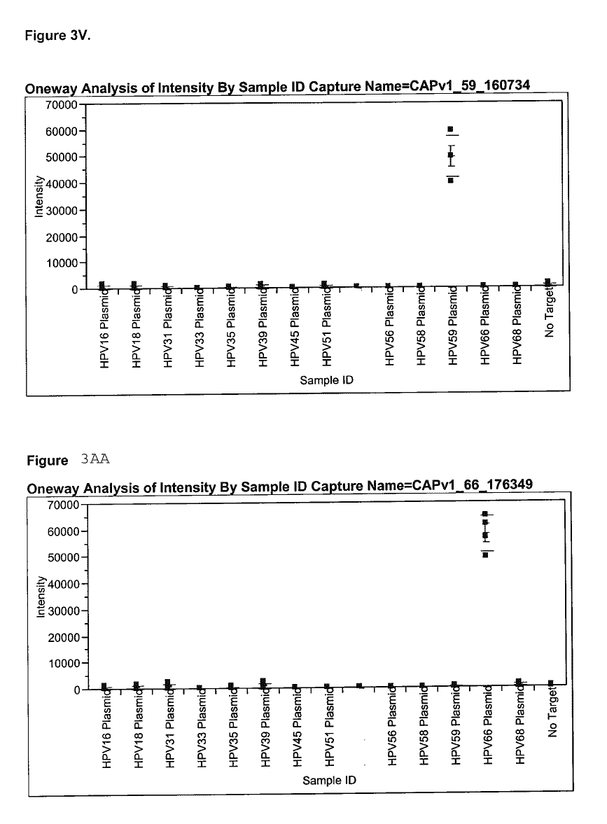

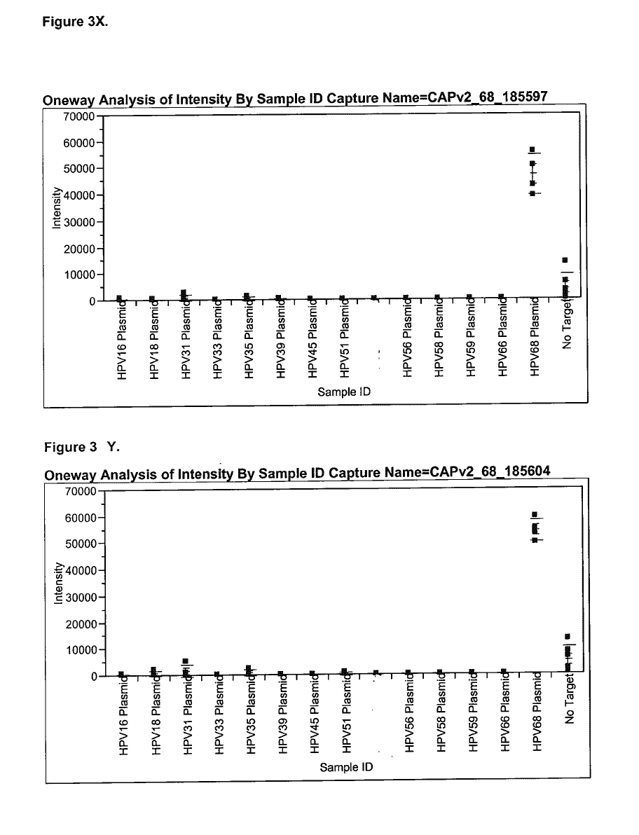

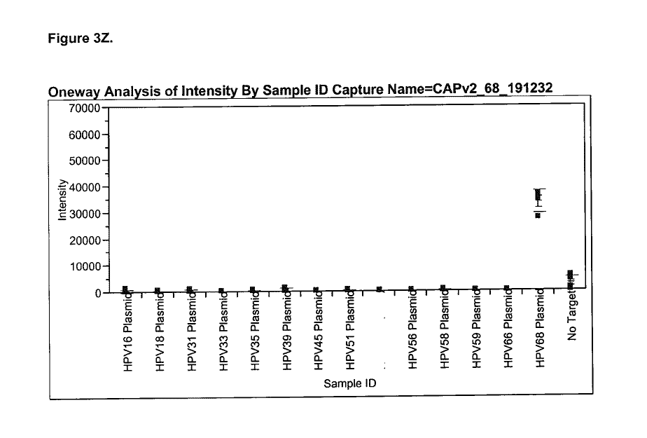

Data from additional studies are shown in FIG. 3. Table 18 summarizes the capture and mediator oligonucleotides and plasmids used for HPV subtypes 16, 18, 31, 33, 35, 39, 45, 51, 56, 58, 59, 66, and 68. Fr1, Fr2, and Fr3 refer to fractional plasmids from Dr. Robert Burk's laboratory at the Albert Einstein College of Medicine (AECOM), Bronx, N.Y.

TABLE 18
Capture and Mediator Oligonucleotides for Plasmid Detection

| | Target | Number of | | |
|---|---|---|---|---|
| HPV Type | Source | Copies | Capture Oligos | Mediator Oligos |
| 16 | ATCC-45113 | 6E7 | CAPv1_16_18971 | MEDv1_16_19232 |
| | | | CAPv2_16_22767 | MEDv2_16_23263 |
| | | | CAPv2_16_23109 | MEDv2_16_22953 |
| 18 | ATCC-45152 | 6E7 | CAPv1_18_38693 | MEDv1_18_38687 |
| | | | CAPv1_18_38772 | |
| 31 | Fr1, Fr2, Fr3- | 6E7 | CAPv1_31_100917 | MEDv1_31_100961 |
| | R. Burk | | CAPv1_31_104028 | MEDv1_31_112000 |
| | (AECOM) | | | |
| 33 | Fr1, Fr2, Fr3- | 6E7 | CAPv1_33_54589 | MEDv1_33_54629 |
| | R. Burk | | CAPv1_33_54824 | MEDv1_33_54876 |
| | (AECOM) | | | |
| 35 | ATCC-40330 | 6E7 | CAPv1_35_117187 | MEDv1_35_125066 |
| | ATCC-40331 | | CAPv1_35_125005 | |
| 39 | Fr1, Fr2-R. | 6E7 | CAPv1_39_205414 | MEDv1_39_205568 |
| | Burk | | CAPv1_39_213212 | |
| | (AECOM) | | | |
| 45 | Fr1, Fr2-R. | 6E7 | CAPv1_45_126880 | MEDv1_45_126936 |
| | Burk | | CAPv1_45_127211 | MEDv1_45_127265 |
| | (AECOM) | | CAPv1_45_128173 | MEDv1_45_128326 |
| 51 | Fr1, Fr2, Fr3- | 6E7 | CAPv1_51_216256 | MEDv1_51_216346 |
| | R. Burk | | CAPv1_51_220901 | MEDv1_51_228853 |
| | (AECOM) | | | |
| 56 | ATCC-40549 | 6E7 | CAPv1_56_142497 | MEDv1_56_142563 |
| | | | CAPv1_56_142511 | |
| 58 | Fr1, Fr2, Fr3- | 6E7 | CAPv1_58_66078 | MEDv1_58_70648 |
| | R. Burk | | CAPv1_58_70587 | MEDv1_58_66290 |
| | (AECOM) | | | MEDv1_58_65889 |
| 59 | Fr1, Fr2, Fr3- | 6E7 | CAPv2_59_158382 | MEDv1_59_158467 |
| | R. Burk | | CAPv2_59_160734 | MEDv1_59_160767 |
| | (AECOM) | | | |
| 66 | Fr1, Fr2, Fr3- | 6E7 | CAPv1_66_176349 | MEDv1_66_176398 |
| | R. Burk | | | |
| | (AECOM) | | | |
| 68 | Fr1, Fr2, Fr3- | 6E7 | CAPv2_68_185597 | MEDv2_68_185707 |
| | R. Burk | | CAPv2_68_185604 | MEDv2_68_185841 |
| | (AECOM) | | CAPv2_68_191232 | MEDv2_68_191269 |

Diluted samples of all plasmids were tested for double-stranded DNA concentration using a Nanodrop Model ND-1000 UV spectrophotometer. Plasmids were diluted to 10 pM concentration prior to testing. Sample-loaded cartridges were tested using DEV1 parameters on Naptune II instruments, with onboard sonication and liquid shuttle parameters set to “0”. 6E7 (60,000,000) plasmid DNA copies were used as targets. Tests were performed in quadruplicate for each target. All assays were imaged on the Verigene Reader with well saturation set to 1%. Each set of relevant plasmid capture replicates is evaluated by the following criteria: A capture must exhibit a ratio of 1.5:1 or higher for mean target capture signal intensity:highest non-target capture signal intensity.

Intensity ratio results for each capture oligonucleotide compared against the nonspecific plasmid with the highest signal are summarized in Table 19. No target intensitites were calculated based on the image captured at the maximum exposure time (2976 msec). Exposure times for plasmid-based detection at 6E7 copes were less than 500 msec in all cases.

TABLE 19
Intensity Ratio Results Applied to Capture Oligonucleotides

| | Mean | Nonspecific | Mean | |
|---|---|---|---|---|
| Capture Oligo | Intensity | Plasmid | Intensity | Ratio |
| CAPv1_16_18973 | 9932.46 | HPV31 | 282.04 | 35.2:1 |
| CAPv1_16_22767 | 15711.2 | HPV18 | 556.6 | 28.2:1 |
| CAPv1_16_23109 | 45287.2 | HPV18 | 1719.9 | 26.3:1 |
| CAPv1_18_38772 | 57730.0 | HPV31 | 703.8 | 82:1 |
| CAPv1_31_100917 | 57060.4 | HPV33 | 425 | 134.3:1 |
| CAPv1_31_104028 | 23845.0 | HPV33 | 227 | 105:1 |
| CAPv1_33_54589 | 53428.7 | HPV35 | 1147.4 | 46.6:1 |
| CAPv1_33_54824 | 41719.1 | HPV35 | 944 | 44.2:1 |
| CAPv1_35_117187 | 39597.6 | HPV39 | 524.7 | 75.5:1 |
| CAPv1_35_125005 | 52151.8 | HPV31 | 791.6 | 65.9:1 |
| CAPv1_39_205414 | 19506.1 | HPV31 | 372.9 | 52.3:1 |
| CAPv1_39_213212 | 55990.2 | HPV31 | 631.2 | 88.7:1 |
| CAPv1_45_126880 | 27449.9 | HPV31 | 837.8 | 32.8:1 |
| CAPv1_45_127211 | 49464.6 | HPV18 | 1129.9 | 43.8:1 |
| CAPv1_45_128173 | 26478.1 | HPV31 | 465.8 | 56.8:1 |
| CAPv1_51_216256 | 53642.6 | HPV39 | 1737.8 | 30.9:1 |
| CAPv1_51_220901 | 43959.8 | HPV39 | 1105 | 39.8:1 |
| CAPv1_56_142497 | 58885.8 | HPV39 | 2315.2 | 25.4:1 |
| CAPv1_56_142511 | 42919.5 | HPV18 | 1067 | 40.2:1 |
| CAPv1_58_66078 | 40428.5 | HPV18 | 593.8 | 68.1:1 |
| CAPv1_58_70587 | 51881.7 | HPV31 | 666.5 | 77.8:1 |
| CAPv1_59_158382 | 54730.6 | HPV39 | 1011.8 | 54.1:1 |
| CAPv1_59_160734 | 49493.2 | HPV16 | 434 | 114:1 |
| CAPv1_66_176349 | 57865.1 | HPV68 | 737.7 | 78.4:1 |
| CAPv2_68_185597 | 46914.2 | HPV31 | 841.6 | 55.7:1 |
| CAPv2_68_185604 | 53936.1 | HPV31 | 1453.4 | 37.1:1 |
| CAPv2_68_191232 | 33334.8 | HPV31 | 519.5 | 64.2:1 |

All of the oligonucleotide capture probes specific for one of the HPV subtypes above demonstrated very high detection specificity at 6E7 copy number of plasmid.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method for detecting high risk human papilloma virus (HPV) in a sample, comprising:
a) providing a substrate having a capture probe bound thereto, wherein at least a portion of the capture probe has a nucleic acid sequence that is complementary to at least a first portion of the genome of a HPV;
b) providing a mediator probe, wherein at least a portion of the mediator probe has a nucleic acid sequence that is complementary to at least a second portion of the HPV genome that is different than the first portion and a nucleotide sequence that is complementary to a non-HPV sequence on oligonucleotides bound to a gold particle, wherein the nucleic acid sequence in the capture probe or the mediator probe, or both, are HPV-subtype specific;
c) contacting a sample suspected of having HPV that is subjected to an amplification reaction with HPV-specific primers, the substrate, the mediator probe and gold particles having oligonucleotides with sequences that are complementary to the nucleotide sequence in the mediator probe under conditions that are effective for the hybridization of the nucleic acid sequence in the capture probe and the nucleic acid sequence in the mediator probe to amplified HPV DNA in the sample and for the hybridization of the nucleotide sequence in the mediator probe to the oligonucleotides bound to the gold particle;
d) washing the substrate to remove non-specifically bound material; and
e) detecting whether gold particles are bound to the substrate, wherein binding of gold particles to the substrate is indicative of the presence of a specific subtype of HPV in the sample.

2. The method of claim 1 wherein the HPV that is detected is HPV16, HPV18, HPV31, HPV33, HPV35, HPV39, HPV45, HPV51, HPV56, HPV58, HPV59, HPV66, or HPV68.

3. The method of claim 1 wherein the HPV-specific primers amplify HPV16, HPV18, HPV31, HPV33, HPV35, HPV39, HPV45, HPV51, HPV56, HPV58, HPV59, HPV66, or HPV68.

4. The method of claim 1 wherein the capture probe includes a nucleic acid sequence corresponding to one of SEQ ID No. 8-22, 38-50, 63-70, 83-94, 103-111, 119-130, 144-154, 168-175, 186-195, 207-225, 239-248, 259-264, or 276-299, or a sequence with at least 90% nucleotide sequence identity thereto.

5. The method of claim 1 wherein the mediator probe includes a nucleic acid sequence corresponding one of SEQ ID No. 23-31, 51-55, 71-78, 95-98, 112-115, 131-135, 155-161, 176-181, 195-199, 226-230, 249-254, 264-267, or 300-313, or a sequence with at least 90% nucleotide sequence identity thereto.

6. The method of claim 4 or 5 wherein the nucleic acid sequence has least 95% nucleotide sequence identity to SEQ ID No. 8-22, 38-50, 63-70, 83-94, 103-111, 119-130, 144-154, 168-175, 186-195, 207-225, 239-248, 259-264, or 276-299 or SEQ ID No. 23-31, 51-55, 71-78, 95-98, 112-115, 131-135, 155-161, 176-181, 195-199, 226-230, 249-254, 264-267, or 300-313.

7. The method of claim 1 wherein the sample is contacted with the mediator probe so as to form a mixture, and the mixture is then contacted with the substrate.

8. The method of claim 1 wherein the sample is contacted with the substrate, and then contacted with the mediator probe.

9. The method of claim 1 wherein the sample is contacted simultaneously with the mediator probe and the substrate.

10. The method of claim 1 wherein the particles are nanoparticles.

11. The method of claim 1 wherein the detecting comprises contacting the substrate with silver stain.

12. The method of claim 1 wherein the detecting comprises detecting light scattered by the particle.

13. The method of claim 1 wherein the detecting comprises observation with an optical scanner.

14. A kit comprising at least two of:
a) a capture probe, at least a portion of which has a nucleic acid sequence that complementary to at least a first portion of a genome of a HPV;
b) a mediator probe, at least a portion of which has a nucleic acid sequence that is complementary to at least a second portion of the genome of the HPV that is different than the first portion and a nucleotide sequence that is complementary to a non-HPV sequence on oligonucleotides bound to a gold particle; or
c) gold particles having the oligonucleotides;
wherein the nucleic acid sequence in the capture probe or the mediator probe has SEQ ID No. 8-31, 38-55, 63-78, 83-98, 103-115, 119-135, 144-161, 168-181, 186-199, 207-230, 239-254, 259-267, or 276-313, or a sequence with at least 90% nucleotide sequence identity thereto.

15. The kit of claim 14 which further comprises a primer having SEQ ID No. 1-7, 32-37, 79-82, 96-102, 116-118, 136-143, 162-167, 182-185, 200-206, 231-238, 255-258, or 268-275 or a sequence with at least 90% nucleotide sequence identity thereto.

16. The kit of claim 14 wherein the capture or mediator probe have a sequence selected from a) SEQ ID No. 8-31 or a sequence with at least 90% nucleotide sequence identity thereto; b) SEQ ID No. 38-55 or a sequence with at least 90% nucleotide sequence identity thereto; c) SEQ ID No. 63-78 or a sequence with at least 90% nucleotide sequence identity thereto; d) SEQ ID No. 83-98 or a sequence with at least 90% nucleotide sequence identity thereto; e) SEQ ID No.103-115 or a sequence with at least 90% nucleotide sequence identity thereto; f) SEQ ID No. 119-135 or a sequence with at least 90% nucleotide sequence identity thereto; g) SEQ ID No. 144-161 or a sequence with at least 90% nucleotide sequence identity thereto; h) SEQ ID No. 168-181 or a sequence with at least 90% nucleotide sequence identity thereto; i) SEQ ID No.186-199 or a sequence with at least 90% nucleotide sequence identity thereto; j) SEQ ID No. 207-230 or a sequence with at least 90% nucleotide sequence identity thereto; k) SEQ ID No. 239-25 or a sequence with at least 90% nucleotide sequence identity thereto; 1) SEQ ID No. 259-267 or a sequence with at least 90% nucleotide sequence identity thereto; or m) SEQ ID No. 276-313 or a sequence with at least 90% nucleotide sequence identity thereto.

17. An isolated oligonucleotide comprising one of SEQ ID Nos. 1-313, a sequence with at least 80% sequence identity thereto, the complement of one of SEQ ID No. 1-313 or the sequence with 80% sequence identity thereto, or a fragment thereof with at least 10 contiguous nucleotides.

18. The isolated oligonucleotide of claim 17 which has no more than 100 nucleotides.

19. The isolated oligonucleotide of claim 17 which has at least 90% sequence identity to one of SEQ ID Nos. 1-313 or the complement thereof, or a fragment thereof with at least 10 contiguous nucleotides.
